(19) United States
(12) Patent Application Publication
(10) Pub. No.: US 2026/0256822 A1
(43) Pub. Date: Sep. 3, 2026

(54) ENTRY-MODULATING AGENTS AND USES THEREFOR

(71) Applicants: Griffith University, Nathan (AU); Swiss Tropical and Public Health Institute, Allschwil (CH); Universitat Basel, Basel (CH)

(72) Inventors: Michael P. Jennings, Nathan (AU); Christopher J. Day, Nathan (AU); Gerd Pluschke, Allschwil (CH)

(21) Appl. No.: 18/878,263

(22) PCT Filed: Jun. 26, 2023
(86) PCT No.: PCT/AU2023/050581

§ 371 Date: Dec. 23, 2024

(30) Foreign Application Priority Data
Jun. 24, 2022 (AU) 2022901757

(51) Int. Cl. A61K 31/7048; A61K 31/473; A61K 31/555; A61K 45/06; A61P 33/06

(52) U.S. Cl. CPC A61K 31/7048; A61K 31/473; A61K 31/555; A61K 45/06; A61P 33/06

(57) ABSTRACT

This disclosure relates generally to the use of compounds that inhibit binding of Plasmodium cysteine-rich protective antigen (CyRPA) to glycans of cells that are susceptible to infection by Plasmodium pathogens, including glycans terminating with α2-6-linked sialic acid, in methods, compositions and kits for inhibiting the interaction of a Plasmodium pathogen to Plasmodium-binding glycan-expressing cells, including α2-6-linked sialic acid-expressing cells such as erythrocytes, and for treating or inhibiting the development of malaria.

### RELATED APPLICATIONS

This application claims priority to Australia Patent Application No. 2022901757 entitled “Entry-modulating agents and uses therefor” filed 24 Jun. 2022, the contents of which are incorporated herein by reference in their entirety.

### FIELD

This disclosure relates generally to the use of compounds that inhibit binding of Plasmodium cysteine-rich protective antigen (CyRPA) to glycans of cells that are susceptible to infection by Plasmodium pathogens, including glycans terminating with α2-6-linked sialic acid, in methods, compositions and kits for inhibiting the interaction of a Plasmodium pathogen to Plasmodium-binding glycan-expressing cells, including α2-6-linked sialic acid-expressing cells such as erythrocytes, and for treating or inhibiting the development of malaria.

### BACKGROUND

Malaria parasite invasion of erythrocytes involves a complex cascade of highly specific molecular interactions between merozoite ligands and host receptors, which mediates recognition, attachment and active entry of parasites into the host cells. While most of the Plasmodium falciparum merozoite surface ligands involved in erythrocyte invasion appear to be functionally redundant (Cowman, Tonkin et al. 2017. Cell Host Microbe 22(2): 232-245), all three components of a ternary invasion complex consisting of P. falciparum cysteine-rich protective antigen (PfCyRPA) (Dreyer, Matile et al. 2012. BMC Biotechnol. 10: 87), P. falciparum reticulocyte binding-like homologous protein 5 (PfRH5) (Baum, Chen et al. 2009. Int J Parasitol 39(3): 371-380) and P. falciparum Rh5 interacting protein (PfRipr) (Chen, Lopaticki et al. 2011. PLoS Pathog 7(9): e1002199) are refractory to genetic disruption (Baum, Chen et al. 2009, supra; Chen, Lopaticki et al. 2011, supra; Reddy, Amlabu et al. 2015. Proc Natl Acad Sci USA 112(4): 1179-1184) and antibodies specific for all three components efficiently block parasite invasion in both in vitro and in vivo models (Chen, Lopaticki et al. 2011, supra; Douglas, Williams et al. 2014, J Immunol 192(1): 245-258; Favuzza, Blaser et al. 2016. MalarJ 15: 161; Favuzza, Guffart et al. 2017. Elife 6; Healer, Chiu et al. 2018. Parasitology 145(7): 839-847). While basigin and SEMA7A function as erythrocyte receptors of PfRH5 and PfRipr, respectively (Crosnier, Bustamante et al. 2011. Nature 480(7378): 534-537; Nagaoka, Kanoi et al. 2020. Sci Rep 10(1): 6573), the exact receptor for PfCyRPA in the ternary invasion complex was not clear prior to the studies described herein. The tridimensional structure of PfCyRPA (Chen, Xu et al. 2017. Elife 6; Favuzza, Guffart et al. 2017, supra) revealed that it adopts a six-bladed β-propeller fold and that its overall structure resembles the catalytic domain of neuraminidases. While PfCyRPA contains an Asp-box as sialidase signature motif, it lacks key residues necessary for catalysis, providing a structural correlate of the absence of enzymatic activity (Dreyer, Matile et al. 2012, supra).

### SUMMARY

The present disclosure is predicated in part on the determination that CyRPA polypeptides from P. falciparum bind to glycans terminating with α2-6-linked sialic acid, which are expressed on the surface of human erythrocytes, and that α2-6 sialidase treatment of erythrocytes reduces binding of CyRPA and parasite multiplication, indicating that α2-6-Neu5Ac lectin activity of CyRPA is important for erythrocyte invasion by Plasmodium parasites. Based on this determination, the present inventors identified compounds that bind to Plasmodium CyRPA and inhibit interaction of the CyRPA with α2-6-linked sialic acid as well as other glycans on the surface or erythrocytes. These findings have been reduced to practice in agents and methods for inhibiting the interaction of Plasmodium CyRPA with Plasmodium CyRPA-binding glycan-expressing cells, including α2-6-Neu5Ac glycan-expressing cells, for inhibiting replication of Plasmodium parasites, and for treating or inhibiting the development of malaria, as described hereafter.

Accordingly, disclosed herein in one aspect are methods for inhibiting interaction of a Plasmodium cysteine-rich protective antigen (CyRPA) with an α2-6-linked sialic acid. These methods generally comprise, consist or consist essentially of contacting the CyRPA with a ligand of the CyRPA which when bound to the CyRPA inhibits interaction of the CyRPA with the α2-6-linked sialic acid. Suitably, the α2-6-linked sialic acid is one expressed by a cell that is capable of infection by a Plasmodium pathogen.

In another aspect, methods are disclosed for inhibiting interaction of a Plasmodium pathogen with an α2-6-linked sialic acid-expressing cell. These methods generally comprise, consist or consist essentially of contacting the Plasmodium species with a ligand of a Plasmodium CyRPA which, when bound to the CyRPA, inhibits interaction of the CyRPA with an α2-6-linked sialic acid of the α2-6-linked sialic acid-expressing cell, thereby inhibiting the interaction of the Plasmodium pathogen with the α2-6-linked sialic acid-expressing cell.

In yet another aspect, methods are disclosed for inhibiting entry of a Plasmodium pathogen into an α2-6-linked sialic acid-expressing cell. These methods generally comprise, consist or consist essentially of contacting the Plasmodium pathogen with a ligand of a Plasmodium CyRPA which, when bound to the CyRPA, inhibits entry of the Plasmodium pathogen into the α2-6-linked sialic acid-expressing cell, thereby inhibiting the interaction of the Plasmodium pathogen with the α2-6-linked sialic acid-expressing cell.

Disclosed herein in another aspect are methods for treating or inhibiting the development of a Plasmodium pathogen infection in a subject. These methods generally comprise, consist or consist essentially of administering to the subject an effective amount of a ligand of a Plasmodium CyRPA which, when bound to the CyRPA, inhibits interaction of the CyRPA with an α2-6-linked sialic acid of an α2-6-linked sialic acid-expressing cell, to thereby treat or inhibit the development of the Plasmodium pathogen infection in the subject. Suitably, the effective amount is one that inhibits entry of the Plasmodium pathogen into the α2-6-linked sialic acid-expressing cell, and/or that inhibits the Plasmodium pathogen invasion of the α2-6-linked sialic acid-expressing cell, and/or that inhibits replication of P. falciparum in the α2-6-linked sialic acid-expressing cell, and/or that inhibits spreading of the Plasmodium pathogen within the subject. In some embodiments, the subject is infected with the Plasmodium pathogen or is identified as having a Plasmodium pathogen infection. In other embodiments, the subject is not infected with the Plasmodium pathogen and in representative examples of this type, the uninfected subject is planning to visit or is visiting a malaria endemic area.

In related aspects, the present disclosure provides compositions for use in therapy or prophylaxis of a Plasmodium pathogen infection in a subject. These compositions generally comprise, consist or consist essentially of a ligand of a Plasmodium CyRPA which, when bound to the CyRPA, inhibits interaction of the CyRPA with an α2-6-linked sialic acid of an α2-6-linked sialic acid-expressing cell, and optionally a pharmaceutically acceptable carrier.

The present disclosure further provides in another aspect kits for use in therapy or prophylaxis of a Plasmodium pathogen infection in a subject. These kits generally comprise, consist or consist essentially of a ligand of a Plasmodium CyRPA which, when bound to the CyRPA, inhibits interaction of the CyRPA with an α2-6-linked sialic acid of an α2-6-linked sialic acid-expressing cell, and optionally instructional material for performing the treatment. In some embodiments, the kits comprise an ancillary agent (e.g., an antimalarial agent) useful for the treatment of a Plasmodium pathogen infection.

In still another aspect, methods are provided for identifying an agent that inhibits interaction of a Plasmodium pathogen with an α2-6-linked sialic acid-expressing cell, or that inhibits entry of a Plasmodium pathogen into an α2-6-linked sialic acid-expressing cell, or that inhibits Plasmodium pathogen invasion of an α2-6-linked sialic acid-expressing cell, or that is useful for treating or inhibiting the development of a Plasmodium pathogen infection. These methods generally comprise: contacting a Plasmodium CyRPA polypeptide with a candidate agent; detecting binding of the candidate agent to the CyRPA polypeptide, and determining whether the candidate agent inhibits binding of the CyRPA polypeptide to an α2-6-linked sialic acid, wherein presence of the inhibition indicates that the candidate agent is an agent that inhibits interaction of a Plasmodium pathogen with an α2-6-linked sialic acid-expressing cell, or that inhibits entry of a Plasmodium pathogen into an α2-6-linked sialic acid-expressing cell, or that inhibits Plasmodium pathogen invasion of an α2-6-linked sialic acid-expressing cell, or that is useful for treating or inhibiting the development of a Plasmodium pathogen infection.

The CyRPA ligands disclosed herein have also been found to bind the CyRPA of Plasmodium pathogens (e.g., P. vivax) that bind glycans other than α2-6-linked sialic acid and are proposed therefore to be useful for inhibiting entry of Plasmodium pathogens generally into Plasmodium CyRPA-binding glycan-expressing cells. Accordingly, in yet another aspect, methods are disclosed herein for inhibiting entry of a Plasmodium pathogen into a Plasmodium CyRPA-binding glycan-expressing cell. These methods generally comprise, consist or consist essentially of contacting the Plasmodium pathogen with a Plasmodium CyRPA-binding ligand disclosed herein, thereby inhibiting the interaction of the Plasmodium pathogen with the Plasmodium CyRPA-binding glycan-expressing cell.

Disclosed herein in a related aspect are methods for treating or inhibiting the development of a Plasmodium pathogen infection in a subject. These methods generally comprise, consist or consist essentially of administering to the subject an effective amount of a Plasmodium CyRPA-binding ligand disclosed herein, to thereby treat or inhibit the development of the Plasmodium pathogen infection in the subject. Suitably, the effective amount is one that inhibits entry of the Plasmodium pathogen into the Plasmodium CyRPA-binding glycan-expressing cell, and/or that inhibits the Plasmodium pathogen invasion of the Plasmodium CyRPA-binding glycan-expressing cell, and/or that inhibits replication of P. falciparum in the Plasmodium CyRPA-binding glycan-expressing cell, and/or that inhibits spreading of the Plasmodium pathogen within the subject. In some embodiments, the subject is infected with the Plasmodium pathogen or is identified as having a Plasmodium pathogen infection. In other embodiments, the subject is not infected with the Plasmodium pathogen and in representative examples of this type, the uninfected subject is planning to visit or is visiting a malaria endemic area.

In any of the aspects disclosed herein, the Plasmodium pathogen is suitably selected from Plasmodium pathogens that infect primates, preferably humans. Representative examples of such Plasmodium pathogens include P. falciparum, P. malariae, P. vivax, P. ovale, P. knowlesi, P. cynomolgi, and P. reichenowi, preferably P. falciparum and P. vivax.

In any of the aspects disclosed herein, the Plasmodium pathogen is a drug-resistant Plasmodium pathogen. In some embodiments, the drug-resistant Plasmodium pathogen is a multidrug-resistant Plasmodium pathogen. In some of the same and other embodiments, the subject is administered a CyRPA-interacting ligand as broadly described above and elsewhere herein when the subject is identified as being infected with a drug-resistant Plasmodium pathogen. In representative embodiments of this type, the subject is administered the CyRPA-interacting ligand after the subject has been administered at least one antimalarial drug to which the Plasmodium pathogen is resistant. The antimalarial drug is suitably a drug that acts directly on a drug sensitive Plasmodium pathogen by killing the drug sensitive Plasmodium pathogen or inhibiting its growth, survival and/or viability.

In any of the aspects disclosed herein, the α2-6-linked sialic acid is suitably a glycan comprising α2-6-Neu5Ac. In preferred embodiments, the glycan comprises α2-6-sialyllactosamine (Neu5Ac-α-(2-6)-Gal-β-(1-4)-GlcNAc).

In any of the aspects disclosed herein, the α2-6-linked sialic acid-expressing cell may be an erythrocyte or hepatocyte, preferably an erythrocyte.

In any of the aspects disclosed herein, the CyRPA ligand may be formulated for oral administration, for enteral administration, for systemic administration or topical administration.

In any of the aspects disclosed herein, the CyRPA ligand may be administered concurrently with an ancillary agent (e.g., an antimalarial agent or adjunctive therapy).

In any of the aspects disclosed herein, the ligand may be a small molecule ligand of CyRPA, preferably selected from the compounds listed in TABLE 1.

TABLE 1

| | |
|---|---|
| 1. A compound of formula (I): | |
| 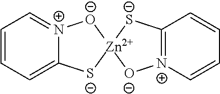 | (I) |
| (Zinc Pyrithione) | |
| 2. A compound of formula (II): | |
| 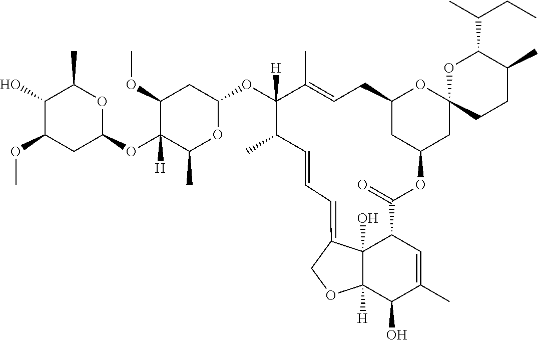 [SMILES: CCC(C)[C@H]1O[C@]2(CC[C@@H]1C)C[C@@H]1C[C@@H](C/C=C(\C)[C@@H](O[C@H]3C[C@H](OC)[C@@H](O[C@H]4C[C@@H](OC)[C@H](O)[C@@H](C)O4)[C@H](C)O3)[C@@H](C)/C=C/C=C3\CO[C@@H]4[C@H](O)C(C)=C[C@@H](C(=O)O1)[C@]34O)O2] | (II) |
| (Ivermectin) | |
| 3. A compound of formula (III): | |
| 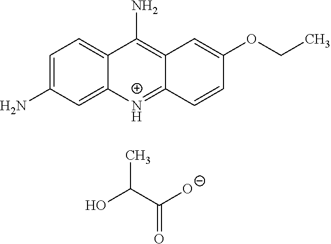 | (III) |
| (RIVANOL) | |

### BRIEF DESCRIPTION OF THE DRAWINGS

FIG. 1 is a diagrammatic representation showing glycan array and SPR analysis of glycan binding of CyRPA. A). Glycan array analysis of 402 glycans of CyRPA from P. falciparum (Pf) and P. reichenowi (Pr). Red indicates binding significantly above background; white indicates no binding. Each thin line represents one glycan, list of glycans is listed in Dataset Si. Mono: monosaccharides; Gal: terminal galactose; terminal GalNAc: N-acetylgalactosamine; Fuc: fucosylated glycans; Neu5Ac: N-acetylneuramic acid containing glycans (sorted by linkage of the sialic acid to the subterminal sugar for clarity); Neu5Gc: N-Glycolylneuramic acid containing glycans; Mann: mannosylated glycans; GlcNAc/chitin terminal and repeating N-acetylglucosamine glycans; Glc: glucose; LMW/HMW GAGs: Low/high molecular weight glycosaminoglycans. B). Equilibrium dissociation constant (K_{D}) of glycans to CyRPA from P. falciparum (Pf) and P. reichenowi (Pr) from SPR analysis.

FIG. 2 is an illustration showing the KD of mutants and the Molecular model of PfCyRPA in complex with mAb C12 and α2-6-SLN. A) Affinities of PfCyRPA wild-type and alanine scanning mutants for the preferred glycan targets (α2-6-SLN-Ac and α2-6-Biant-Ac) and the mAb C12. Red and yellow indicate mutants in lectin site 1 and site 2, respectively; orange represents the alanine exchange mutant at position E148, which is part of both binding sites. B) Molecular model of PfCyRPA in complex with mAb C12 and docked α2-6-SLN-Ac using the X-ray crystal structure of PfCyRPA PDB 5EZN (10.2210/pdb5EZN/pdb)¹¹. PfCyRPA blades B1-B6, the lectin site 2 (yellow) as well as the RipR (purple) and Rh5 (orange) docking domains are marked. C) Molecular model of PfCyRPA in complex with mAb C12 and α2-6-SLN with a 90⁰ tilted view with the key glycan binding residues for site 1 (red) and site 2 (yellow) highlighted. A detailed view of the α2-6-SLN-Ac interaction with PfCyRPA is shown on the left. Amino acids essential for glycan coordination in site 1 are labelled and hydrogen bonds are marked (yellow lines).

FIG. 3 is an illustration showing the binding of glycans to truncated fragments of PfCyRPA. Binding of α2-6-SLN-Ac, α2-6-SLN-Gc and α2-6-Biant-Ac to PfCyRPA fragments anchored onto the surface of fixed HEK cells. The positions of amino acid residues implicated in lectin activity of fragment 2 are marked.

FIG. 4 is a table showing the affinity changes caused by point mutations of PfCyRPA Neu5Ac binding sites 1 and 2. Red indicates residues in site 1 that are highlighted in FIG. 2. Yellow indicates residues in site 2 that are highlighted in FIG. 2. Orange indicates a residue involved in both binding site 1 and 2 that is highlighted in FIG. 2. White indicates residues that map into site 1 of PfCyRPA but are not indicated on FIG. 2

FIG. 5 is a diagrammatic and tabular representation showing the binding of glycans to truncated fragments of PfCyRPA. Binding of α2-6-SLN-Ac, α2-6-SLN-Gc and α2-6-Biant-Ac to PfCyRPA fragments anchored onto the surface of fixed HEK cells. The positions of amino acid residues implicated in lectin activity of fragment 2 are marked in the fragment 2.

FIG. 6 is a graphical representation showing parasite growth inhibition by α2-6-Biant-Ac, treatment of erythrocytes with an α2-6 specific sialidase or by anti-PfCyRPA mAb C12. Inhibition of in vitro P. falciparum parasite growth by A) soluble α2-6-Biant-Ac, C) treatment of human erythrocytes with an α2-6-linkage-specific sialidase in the presence of its cofactor (18P244ST+CMP), and E) by the anti-PfCyRPA mAb C12. B) Lack of growth inhibition by α2-6-Biant-Gc. D) Inhibition of α2-6-Biant-Ac binding to PfCyRPA by mAb C12 determined by analysis of mAb C12 alone, α2-6-Biant-Ac alone and the competition between α2-6-Biant-Ac and mAb C12 for the binding of PfCyRPA. F) Inhibition of P. falciparum growth by mAb C12 in NOD-scid IL2Rγ^{null} mice engrafted with human erythrocytes.

FIG. 7 is a graphical representation showing EC₅₀ of PfCyRPA binding compounds in blood stage parasite growth inhibition assays. EC₅₀ values were determined in vitro by measuring incorporation of the nucleic acid precursor [³H]-hypoxanthine by the P. falciparum 3D7 clone. Results of four to eight determinations were cumulated and sigmoidal dose-response curve were fitted to the relationship between log₁₀(compound concentration) and % inhibition.

FIG. 8 is a graphical and diagrammatic representation showing that zinc pyrithione reduces parasitemia in a humanized NSG mouse model for P. falciparum malaria. P. falciparum-infected NSG mice received four i.p. injections of either 5 mg/kg or 10 mg/kg zinc pyrithione on days one to five after infection. Values shown are the mean parasitemia in the peripheral blood of two mice per group.

FIG. 9 is a graphical representation showing that zinc pyrithione reduces parasitemia in a humanized blood-stage NSG mouse P. falciparum blood-stage malaria model P. falciparum-infected humanized NSG mice received four i.p. injections of either 5 mg/kg or 10 mg/kg Zinc pyrithione on days one to five after infection. Values are the mean parasitemia in peripheral blood of two mice per group.

FIG. 10 is a graphical representation showing standard curves of zinc pyrithione, pyrithione and zinc using CyRPA and NDM for detection of zinc pyrithione levels in mouse serum. Standard curves of spiked normal mouse serum with ZnPT, Zinc sulfate and pyrithione with two separate ZnPT binding proteins, CyRPA and NDM2. Saturated binding is present by 10 μM for ZnPT, 10 mM for Zinc and 1 mM for pyrithione.

FIG. 11 is a graphical representation showing standard curves of zinc pyrithione, pyrithione and zinc using CyRPA and NDM for detection of zinc pyrithione levels in human serum. Standard curves of spiked normal human serum with ZnPT, Zinc sulfate and pyrithione with two separate ZnPT binding proteins, CyRPA and NDM2. Saturated binding is present by 10 μM for ZnPT, 10 mM for Zinc and 1 mM for pyrithione.

FIG. 12 is a graphical representation showing that Ivermectin reduces parasitemia in a humanized NSG mouse P. falciparum blood-stage malaria model. P. falciparum-infected humanized NSG mice received five i.p. injections of 5 mg/kg Ivermectin on days one to six after infection. Values are the mean parasitemia in peripheral blood of two mice per group.

FIG. 13 is a tabular representation showing the glycan array result of CyRPA. Red indicates binding. Binding is determined by positive interaction in three replicate array experiments. Positive interactions are determined by a background subtracted fluorescence value significantly above background subtracted fluorescence of negative control spots (average background fluorescence from 20 spots+3 standard deviations).

Some figures and text contain color representations or entities. Color illustrations are available from the Applicant upon request or from an appropriate Patent Office. A fee may be imposed if obtained from a Patent Office.

### DETAILED DESCRIPTION

### 1. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

As used herein, the term “about” usually means within an acceptable error range for the type of value and method of measurement. For example, it can mean within 20%, more preferably within 10%, and most preferably still within 5% of a given value or range. Alternatively, especially in biological systems, the term “about” means within about a log (i.e., an order of magnitude) preferably within a factor of two of a given value.

As used herein, “and/or” refers to and encompasses any and all possible combinations of one or more of the associated listed items, as well as the lack of combinations when interpreted in the alternative (or).

The terms “administration concurrently” or “administering concurrently” or “co-administering” and the like refer to the administration of a single composition containing two or more agents, or the administration of each agent as separate compositions and/or delivered by separate routes either contemporaneously or simultaneously or sequentially within a short enough period of time that the effective result is equivalent to that obtained when all such agents are administered as a single composition. By “simultaneously” is meant that the agents are administered at substantially the same time, and desirably together in the same composition. By “contemporaneously” it is meant that the agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The agents may be administered in either order. The term “sequentially” as used herein means that the agents are administered in sequence, for example at an interval or intervals of minutes, hours, days or weeks. If appropriate the agents may be administered in a regular repeating cycle.

The term “agent” includes a compound that induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “agent” includes a cell that is capable of producing and secreting a polypeptide as well as a polynucleotide comprising a nucleotide sequence that encodes that polypeptide. Thus, the term “agent” extends to nucleic acid constructs including vectors such as viral or non-viral vectors, expression vectors and plasmids for expression in and secretion in a range of cells.

The term “ancillary agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “ancillary agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “ancillary agent” includes a cell that is capable of producing and secreting a polypeptide as well as a polynucleotide comprising a nucleotide sequence that encodes that polypeptide.

The terms “antimalarial”, “antimalarial agent” and “antimalarial drug” are used interchangeably herein to mean any and all compounds that are currently known or will be known in the future to terminate, reduce, or prevent infections with mosquito-transmitted intracellular parasites of the genus Plasmodium. Exemplary antimalarial agents include but are not limited to hydroxycloroquine (PLAUENIL), chloroquine (ARALEN), primaquine (PRIMACIN), artemisinin (QINGHAOSU), atovaquone/proguanil (MEPRON), mefloquine (LARIUM), quinicrine (ATABRINE) and doxycycline (GENRX).

As used herein, the term “complex” refers to an assemblage or aggregate of molecules (e.g., peptides, polypeptides, etc.) in direct and/or indirect contact with one another. In specific embodiments, “contact”, or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such embodiments, a complex of molecules (e.g., a peptide and polypeptide) is formed under conditions such that the complex is thermodynamically favored (e.g., compared to a non-aggregated, or non-complexed, state of its component molecules). As used herein the term “complex”, unless described as otherwise, refers to the assemblage of two or more molecules (e.g., peptides, polypeptides or a combination thereof). In specific embodiments, the term “complex” refers to the assemblage of two or three polypeptides.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “drug-resistant Plasmodium pathogen” refers to a Plasmodium pathogen that is resistant to at least one anti-malarial drug that acts directly on the Plasmodium pathogen by killing the Plasmodium pathogen or inhibiting its growth, survival and/or viability. In this context, such a pathogen-directed anti-malarial drug is distinguished from the presently disclosed agents that interfere with host mechanisms that are required by a Plasmodium pathogen for host cell entry and productive replication or persistence, particularly interfering with the interaction between a Plasmodium pathogen CyRPA and a host cell glycan required for Plasmodium pathogen entry and infection of the host cell. It is proposed that the host cell entry inhibitors of the present disclosure are less likely to result in resistance by a Plasmodium pathogen because resistance would require the pathogen to use an alternative host receptor to bind to and infect the host cell, or to become less dependent on the targeted host cell receptor, which would likely require considerable mutational changes in the pathogen. Because it is less likely that Plasmodium pathogens will mutate to target an alternative host cell receptor that is critical for their infection of the host, the chance of generating drug-resistant Plasmodium pathogen mutants with the entry inhibitor approaches disclosed herein is minimized. Accordingly, the presently disclosed host cell entry inhibitors are useful to address infections by Plasmodium pathogens that have developed resistance to traditional drugs. In some embodiments, the drug-resistant Plasmodium pathogen is chloroquine-resistant. In other embodiments, the drug-resistant Plasmodium pathogen is primaquine-resistant. In some embodiments, the drug-resistant Plasmodium pathogen is artemisinin-resistant. In some embodiments, the drug-resistant Plasmodium pathogen is doxycycline-resistant. In yet other embodiments, the drug-resistant Plasmodium pathogen is atovaquone-resistant. In some embodiments, the drug-resistant Plasmodium pathogen is mefloquine-resistant. In further embodiments, the drug-resistant Plasmodium pathogen is resistant to more than one anti-malarial drug. In some embodiments, the drug-resistant pathogen is resistant to atovaquone and proguanil hydrochloride.

By “effective amount” or “therapeutically effective amount”, in the context of treating or preventing a condition, is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials. Non-limiting symptoms of malaria include impaired consciousness, deep breathing and respiratory distress, clinical jaundice and evidence of vital organ dysfunction, shaking chills that can range from moderate to severe, high fever, profuse sweating, headache, nausea, vomiting, abdominal pain, diarrhea, anemia, muscle pain, convulsions, coma and bloody stools.

The terms “entry inhibitor”, “cell-entry inhibitor” and “host cell entry inhibitor” are used interchangeable herein to refer to a particular class of drugs that inhibit the ability of a pathogen such a parasite (e.g., a Plasmodium pathogen such as P. falciparum) to successfully enter and thereby infect a target cell (e.g., an erythrocyte).

The term “expression”, or its grammatical equivalents, refers to the cellular processes involved in producing RNA and proteins, including where applicable, for example, transcription, transcript processing, translation and protein folding, modification and processing, secretion of protein and localization of protein to a cellular component (e.g., cell surface, cytoplasm, nucleus etc.).

As used herein, the term “glycan” refers to polymers made up of sugar monomers, typically joined by glycosidic bonds also referred to herein as linkages. Glycans can be homo- or heteropolymers of monosaccharide residues, and can be linear or branched. Glycans can be found attached to proteins as in glycoproteins and proteoglycans. In general, they are found on the exterior surface of cells. O- and N-linked glycans are very common in eukaryotes but may also be found, although less commonly, in prokaryotes. The term “glycan” may also be used to refer to the carbohydrate portion of a glycoconjugate, such as a glycoprotein, glycolipid, or a proteoglycan, even if the carbohydrate is only an oligosaccharide. Common monomers of glycans include, but are not limited to trioses, tetroses, pentoses, glucose, fructose, galactose, xylose, arabinose, lyxose, allose, altrose, mannose, gulose, iodose, ribose, mannoheptulose, sedoheptulose and talose. Amino sugars may also be monomers within a glycan. Glycans comprising such sugars are herein referred to as aminoglycans. Amino sugars, as used herein, are sugar molecules that comprise an amine group in place of a hydroxyl group, or in some embodiments, a sugar derived from such a sugar. Examples of amino sugars include, but are not limited to glucosamine, galactosamine, N-acetylglucosamine, N-acetylgalactosamine, sialic acids (including, but not limited to, N-acetylneuraminic acid and N-glycolylneuraminic acid) and L-daunosamine.

The term “interaction”, including its grammatical equivalents, when referring to an interaction between two molecules, refers to the physical contact of the molecules with one another. Generally, such an interaction results in an activity (which produces a biological effect) of one or both of said molecules. The physical contact typically requires binding or association of the molecules with one another and may involve the formation of an induced magnetic field or paramagnetic field, covalent bond formation, ionic interaction (such as, for example, as occurs in an ionic lattice), a hydrogen bond, or alternatively, a van der Waals interaction such as, for example, a dipole-dipole interaction, dipole-induced dipole interaction, induced dipole-induced dipole interaction, or a repulsive interaction, or any combination of the above forces of attraction.

It is to be understood that definitions given to the variables of the generic formulae described herein will result in molecular structures that are in agreement with standard organic chemistry definitions and atom valences.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the present disclosure include, but are not restricted to, any member of the subphylum Chordata including primates (e.g., humans, monkeys and apes, and includes species of monkeys such as from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (e.g., snakes, frogs, lizards etc.), and fish. In specific embodiments, the subject is a primate such as a human in need of treating or inhibiting the development of malaria. However, it will be understood that the terms “patient,” “subject,” “host” or “individual” do not imply that symptoms are present.

The term “Plasmodium” is given its customary taxonomical meaning. Alternative terms used interchangeably herein include “Plasmodium parasite”, “Plasmodium pathogen” or just “Plasmodium” to refer to a microorganism within this genus. Examples of Plasmodium species include Plasmodium falciparum, Plasmodium vivax, Plasmodium malariae, Plasmodium ovale, Plasmodium knowlesi, Plasmodium cynomolgi and Plasmodium reichenowi.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Generally, a pharmaceutically acceptable carrier is substantially nontoxic and non-inflammatory in subjects. Carriers may include excipients and other additives such as diluents, detergents, coloring agents, wetting or emulsifying agents, pH buffering agents, preservatives, transfection agents and the like. In some embodiments, pharmaceutically acceptable carriers are vehicles capable of suspending and/or dissolving active agents. Carriers may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary carriers include, but are not limited to: butylated hydroxytoluene (BHT), calcium carbonate, calcium phosphate (dibasic), calcium stearate, Croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C and xylitol.

The term “unit dosage forms” refers to physically discrete units suitable as unitary dosages for human subjects and other mammals, each unit containing a predetermined quantity of active material calculated to produce the desired therapeutic effect, in association with a suitable pharmaceutically acceptable carrier. The predetermined quantity of the active agent may be the amount prescribed for a single dose (i.e., an amount expected to correlate with a desired outcome when administered as part of a therapeutic regimen) or a fraction thereof. One of ordinary skill in the art will appreciate that the total amount of a therapeutic composition or agent administered to a particular subject is determined by one or more attending physicians and may involve administration of multiple unit dosage forms.

As used herein, the term “sialic acid” refers to a group of molecules where the common molecule includes N-acetyl-5-neuraminic acid (Neu5Ac) having the basic 9-carbon neuraminic acid core modified at the 5-carbon position with an attached acetyl group. Common derivatives of Neu5Ac at the 5-carbon position include: 2-keto-3-deoxy-d-glycero-dgalactonononic acid (KDN) which possesses a hydroxyl group in place of the acetyl group; de-N-acetylation of the 5-N-acetyl group produces neuraminic (Neu); hydroxylation of the S—N-acetyl group produces N-glycolylneuraminic acid (Neu5Gc). The hydroxyl groups at positions 4-, 7-, 8- and 9- of these four molecules (Neu5Ac, KDN, Neu and Neu5Gc) can be further substituted with O-acetyl, O-methyl, O-sulfate and phosphate groups to enlarge this group of compounds. Furthermore, unsaturated and dehydro forms of sialic acids are known to exist.

As used herein a “small molecule” refers to a compound that has a molecular weight of less than 3 kilodaltons (kDa), and typically less than 1.5 kilodaltons, and suitably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the disclosure to identify compounds that modulate a bioactivity. A “small organic molecule” is an organic compound (or organic compound complexed with an inorganic compound (e.g., metal)) that has a molecular weight of less than 3 kilodaltons, less than 1.5 kilodaltons, less than about 1 kDa or even less than about 0.5 kDa.

As used herein, the terms “treatment”, “treating”, and the like, refer to obtaining a desired pharmacologic and/or physiologic effect. The effect may be therapeutic in terms of a partial or complete cure for a disease or condition (e.g., a P. falciparum infection) and/or adverse effect attributable to the disease or condition. These terms also cover any treatment of a condition or disease in a mammal, particularly in a human, and include: (a) inhibiting the disease or condition, i.e., arresting its development; or (b) relieving the disease or condition, i.e., causing regression of the disease or condition; or (c) slowing the progression of the disease or condition.

Each embodiment described herein is to be applied mutatis mutandis to each and every embodiment unless specifically stated otherwise.

### 2. Compounds and Compositions for Modulating P. falciparum Interactions with Glycan-Expressing Cells

The present disclosure is based in part on the identification of compounds that bind to Plasmodium CyRPA (PCyRPA), including P. falciparum CyRPA (PfCyRPA) and P. vivax CyRPA (PvCyRPA), and that inhibit the interaction of the CyRPA with Plasmodium CyRPA-binding glycan-expressing cells, including α2-6-linked sialic acid-expressing cells such as erythrocytes, to thereby inhibit entry of Plasmodium pathogens into those cells with consequential inhibition of Plasmodium pathogen replication. Thus, the compounds of the present disclosure have utility as Plasmodium pathogen entry inhibitors, as inhibitors of Plasmodium pathogen replication and/or persistence, and for treating or inhibiting the development of malaria.

### 2.1 Inhibitors of PfCyRPA—α2-6-Linked Sialic Acid Interaction

The methods and compositions of the present disclosure feature compounds that bind to PCyRPA (i.e., PCyRPA ligand) and inhibit the interaction between PCyRPA and a glycan, including a glycan comprising and/or terminating with an α2-6-linked sialic acid, which glycan is expressed by certain cells, particularly cells that are capable of being infected by Plasmodium pathogens such as erythrocytes and hepatocytes.

In some embodiments, the PCyRPA ligand is Zinc Pyrithione, which is suitably represented by formula (I):

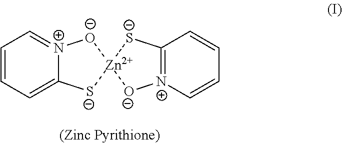

In other embodiments, the PCyRPA ligand is Ivermectin (STROMECTOL), which is suitably represented by formula (II):

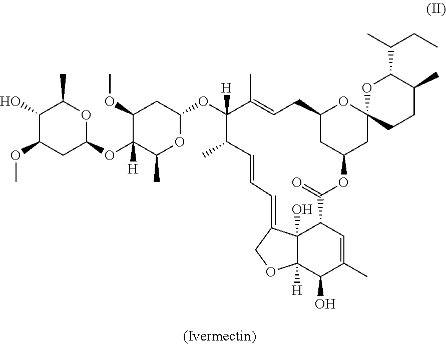 [SMILES: CCC(C)[C@H]1O[C@]2(CC[C@@H]1C)C[C@@H]1C[C@@H](C/C=C(\C)[C@@H](O[C@H]3C[C@H](OC)[C@@H](O[C@H]4C[C@@H](OC)[C@H](O)[C@@H](C)O4)[C@H](C)O3)[C@@H](C)/C=C/C=C3\CO[C@@H]4[C@H](O)C(C)=C[C@@H](C(=O)O1)[C@]34O)O2]

In still other embodiments, the PCyRPA ligand is Ethacridine Lactate Monohydrate (RIVANOL), which is suitably represented by formula (III):

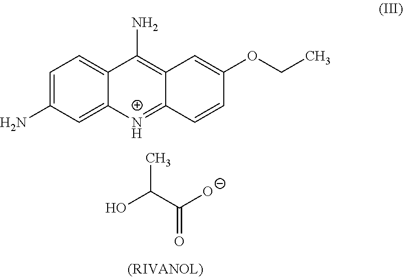

It will be appreciated that the structures of compounds disclosed herein may include asymmetric centers, including asymmetric carbon atoms. It is to be understood accordingly that the isomers arising from such asymmetry (e.g., all enantiomers, stereoisomers, rotamers, tautomers, diastereomers, or racemates) are included within the scope of the disclosure. Such isomers can be obtained in substantially pure form by classical separation techniques or by stereochemically controlled synthesis. Furthermore, the structures and other compounds and moieties discussed in this application also include all tautomers thereof.

Compounds of formulas (I), (II) and (III) as described herein may be purchased from commercial sources such a chemical manufacturers or suppliers well known to the skilled person. Alternatively, the compounds may be synthesized from commercially available starting materials and/or synthetic intermediates using art recognized synthetic routes. Many of the compounds described are known drug molecules, also referred to as active pharmaceutical ingredients (APIs), and have received regulatory approval for alternative indications to those described herein. Synthetic routes to drug molecules, such as those encompassed by compounds of formulas (I), (II) and (III), are known to the skilled person.

### 3. Screening Assays

The present disclosure further encompasses the use of a PCyRPA polypeptide in screening assays for identifying agents that inhibit interaction of a Plasmodium pathogen with an α2-6-linked sialic acid-expressing cell, or that inhibit entry of a Plasmodium pathogen into an α2-6-linked sialic acid-expressing cell, or that are useful for treating or inhibiting the development of a Plasmodium pathogen infection and malaria. Thus in certain aspects, the present disclosure relates to the use of a PCyRPA polypeptide to identify compounds (agents) which are antagonists of a PCyRPA:glycan interaction/complex including a PCyRPA:α2-6-linked sialic acid interaction/complex. Compounds identified through this screening can be tested to assess their ability to antagonize the binding of a Plasmodium pathogen to a glycan-expressing cell such as an α2-6-linked sialic acid-expressing cell and/or inhibit entry of a Plasmodium pathogen into a glycan-expressing cell such as an α2-6-linked sialic acid-expressing cell. Optionally, these compounds can further be tested in animal models to assess their ability to inhibit or treat a Plasmodium pathogen infection and malaria.

There are numerous approaches to screening for therapeutic agents for inhibiting Plasmodium pathogen infection by targeting a PCyRPA:glycan interaction/complex including a PCyRPA:α2-6-linked sialic acid interaction/complex. In certain embodiments, high-throughput screening of compounds can be carried out to identify agents that perturb this interaction. In certain embodiments, the assay is carried out to screen and identify compounds that specifically inhibit or reduce binding of a PCyRPA polypeptide to an α2-6-linked sialic acid or α2-6-linked sialic acid-expressing cell. In a further embodiment, the compounds can be identified by their ability to interact with a PCyRPA polypeptide.

The PCyRPA polypeptide may comprise a precursor PCyRPA polypeptide and biologically active fragments thereof, which interacts with an α2-6-linked sialic acid or α2-6-linked sialic acid-expressing cell, as defined herein. Non-limiting examples of such biologically active fragments include processed forms of a precursor PCyRPA polypeptide such as but not limited to amino acid sequences comprising a mature PCyRPA amino acid sequence.

Representative precursor PCyRPA polypeptides include precursor PfCyRPA polypeptides and precursor PvCyRPA polypeptides, non-limiting examples of which comprise, consist or consist essentially of an amino acid sequence selected from:

[SEQ ID NO: 1; PfCyRPA precursor polypeptide]
MIIPFHKKFISFFQIVLVVLLLCRSINCDSRHVFIRTELSFIKNN
VPCIRDMFFIYKRELYNICLDDLKGEEDETHIYVQKKVKDSWITL
NDLFKETDLTGRPHIFAYVDVEEIIILLCEDEEFSNRKKDMTCHR
FYSNDGKEYNNSEITISDYILKDKLLSSYVSLPLKIENREYFLIC
GVSPYKFKDDNKKDDILCMASHDKGETWGTKIVIKYDNYKLGVQY
FFLRPYISKNDLSFHFYVGDNINNVKNVNFIECTHEKDLEFVCSN
RDFLKDNKVLQDVSTLNDEYIVSYGNDNNFAECYIFFNNENSILI
KPEKYGNTTAGCYGGTFVKIDENRTLFIYSSSQGIYNIHTIYYAN
YE;
and
[SEQ ID NO: 2; PvCyRPA precursor polypeptide]
MIVTKIAIFLFFFLRCLSTNTQSKNIIILNDEITTIKSPIHCITD
IYFLFRNELYKTCIQHVIKARTEIHVLVQKKINSTWQTQTKLFED
NMWFELPSVFNFIHNDEIIIVICRYKQKSKRKETICERWNSVTGT
IYKKEDVQIDKEAFANKNLDSYQSVPLTVKNKKFLLICGILSYEY
ETPNKDNFISCVASEDKGRTWGTKILINYEELQKGVPYFYLRPII
FGDEFGFYFYSRISTNNTARGGNYMTCTLEEPNEGKKEYKFKCKH
VSLIKPDKSLQNVTKLNGYYITSYVKKDNFNECYLYYTEQNAIVV
KPKVQNDDLNGCYGGSFVKLDESKALFIYSTGYGVQNIHTLYYTR
YD.

Illustrative PCyRPA biologically active fragments include mature PfCyRPA polypeptides and mature PvCyRPA polypeptides, non-limiting examples of which comprise, consist or consist essentially of an amino acid sequence selected from:

[SEQ ID NO: 3; PfCyRPA mature polypeptide];
DSRHVFIRTELSFIKNNVPCIRDMFFIYKRELYNICLDDLKGEED
ETHIYVQKKVKDSWITLNDLFKETDLTGRPHIFAYVDVEEIIILL
CEDEEFSNRKKDMTCHRFYSNDGKEYNNSEITISDYILKDKLLSS
YVSLPLKIENREYFLICGVSPYKFKDDNKKDDILCMASHDKGETW
GTKIVIKYDNYKLGVQYFFLRPYISKNDLSFHFYVGDNINNVKNV
NFIECTHEKDLEFVCSNRDFLKDNKVLQDVSTLNDEYIVSYGNDN
NFAECYIFFNNENSILIKPEKYGNTTAGCYGGTFVKIDENRTLFI
YSSSQGIYNIHTIYYANYE
and
[SEQ ID NO: 4; PvCyRPA mature polypeptide]
KNIIILNDEITTIKSPIHCITDIYFLFRNELYKTCIQHVIKARTE
IHVLVQKKINSTWQTQTKLFEDNMWFELPSVFNFIHNDEIIIVIC
RYKQKSKRKETICERWNSVTGTIYKKEDVQIDKEAFANKNLDSYQ
SVPLTVKNKKFLLICGILSYEYETPNKDNFISCVASEDKGRTWGT
KILINYEELQKGVPYFYLRPIIFGDEFGFYFYSRISTNNTARGGN
YMTCTLEEPNEGKKEYKFKCKHVSLIKPDKSLQNVTKLNGYYITS
YVKKDNFNECYLYYTEQNAIVVKPKVQNDDLNGCYGGSFVKLDES
KALFIYSTGYGVQNIHTLYYTRYD

The PCyRPA polypeptide may be conveniently prepared by recombinant techniques. For example, the PCyRPA polypeptide may be prepared by a procedure including the steps of: (a) preparing a construct comprising a coding sequence for the PCyRPA polypeptide, wherein the coding sequence is operably connected to a promoter; (b) introducing the construct into a host cell in which the promoter is operable; (c) culturing the host cell to express the coding sequence to thereby produce the encoded polypeptide; and (d) isolating the encoded polypeptide from the host cell. Representative PCyRPA polypeptide coding sequences are known in the art.

Recombinant polypeptides can be conveniently prepared using standard protocols as described for example in Ausubel et al. “Current Protocols in Molecular Biology”, John Wiley & Sons Inc, 1994-2003.

In specific embodiments, the α2-6-linked sialic acid is a glycan comprising α2-6-Neu5Ac, preferably α2-6-sialyllactosamine (Neu5Ac-α-(2-6)-Gal-β-(1-4)-GlcNAc; “2-6SLN”).

A variety of assay formats will suffice and, in light of the present disclosure, those not expressly described herein will nevertheless be comprehended by one of ordinary skill in the art. As described herein, the test or ‘candidate’ compounds (agents) of the present disclosure may be created by any combinatorial chemical method. Alternatively, the compounds may be naturally occurring molecules that are extracted and purified from a suitable source, or synthesized in vivo or in vitro. Compounds (agents) to be tested can be produced, for example, by bacteria, yeast, plants or other organisms (e.g., natural products), produced chemically (e.g., small molecules, including peptidomimetics), or produced recombinantly. Test compounds contemplated by the present disclosure include non-peptidyl organic molecules, peptides, polypeptides, peptidomimetics, sugars, hormones, and nucleic acid molecules. In a specific embodiment, the test agent is a small organic molecule having a molecular weight of less than about 2,000 Daltons.

The test compounds can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase (GST), photoactivatable cross linkers or any combinations thereof.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of a candidate drug on the molecular target as may be manifest in an alteration of binding affinity between a PCyRPA polypeptide and an α2-6-linked sialic acid or α2-6-linked sialic acid-expressing cell.

Candidate compounds may also be selected by electronic screening of well-known large compound libraries, such as the Available Chemical Directory (ACD; http://www.organicworldwide.net/content/available-chemical-directory). Compounds of such libraries may be analyzed by docking programs. In particular, to evaluate the quality of fit and strength of interactions between ligands or potential ligands and a PCyRPA polypeptide, docking programs such as Autodock (available from Oxford Molecular, Oxford, UK), Dock (available from Molecular Design Institute, University of California San Francisco, Calif.), Gold (available from Cambridge Crystallographic Data Centre, Cambridge, UK) and FlexX and FlexiDock (both available from Tripos, St. Louis, Mo.) may be used. These programs and the program Affinity (available from Molecular Simulations, San Diego, Calif.) may also be used in further development and optimization of candidate compounds. Standard molecular mechanics force fields such as CHARMm and AMBER may be used in energy minimization and molecular dynamics.

Merely to illustrate, in an exemplary screening assay of the present disclosure, a compound of interest is contacted with a PCyRPA polypeptide which is ordinarily capable of binding to an to an α2-6-linked sialic acid (e.g. 2-6SLN) or α2-6-linked sialic acid (e.g. 2-6SLN)-expressing cell, and any binding of the compound to the PCyRPA polypeptide is detected to determine whether the compound can form a complex with the PCyRPA polypeptide. Detection and quantification of complex formation and assessment of the affinity of binding between the compound and the PCyRPA polypeptide provides a means for determining the compound's efficacy at inhibiting the binding of the PCyRPA polypeptide to the α2-6-linked sialic acid or α2-6-linked sialic acid-expressing cell. The efficacy of the compound can be assessed by generating dose response curves from data obtained using various concentrations of the test compound.

In another embodiment of a screening assay of the present disclosure, the compound of interest is contacted with a PCyRPA polypeptide which is ordinarily capable of binding to an α2-6-linked sialic acid (e.g. 2-6SLN) or α2-6-linked sialic acid (e.g. 2-6SLN)-expressing cell. To the mixture of the compound and PCyRPA polypeptide is then added a composition containing an α2-6-linked sialic acid (e.g. 2-6SLN). Detection and quantification of PCyRPA polypeptide/α2-6-linked sialic acid complexes provides a means for determining the compound's efficacy at inhibiting complex formation between the PCyRPA polypeptide and the α2-6-linked sialic acid (e.g. 2-6SLN). The efficacy of the compound can be assessed by generating dose response curves from data obtained using various concentrations of the test compound. Moreover, a control assay can also be performed to provide a baseline for comparison. For example, in a control assay, an isolated and/or purified α2-6-linked sialic acid (e.g. 2-6SLN) is added to a composition containing an isolated and/or purified PCyRPA polypeptide, and the formation of PCyRPA polypeptide/α2-6-linked sialic acid complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

The capacity of a compound to modulate the interaction between the PCyRPA polypeptide and an α2-6-linked sialic acid (e.g. 2-6SLN) may be tested by any method which is known to the person of skills in the art to be suitable for assessing the interaction between two proteins. These methods include such technique as e.g., immunoblotting, immunoprecipitation analyses, fluorescence polarization, FRET (Fluorescence Resonance Energy Transfer), BRET (Bioluminescence Resonance Energy Transfer), AlphaScreen™ (Amplified Luminescent Proximity Homogeneous Assay), Scintillation Proximity Assay, ELISA (Enzyme-Linked Immunosorbent Assay), SPR (Surface Plasmon Resonance, also known as BIAcore™), isothermal titration calorimetry (ITC), differential scanning calorimetry, microscale thermophoresis, gel electrophoresis, and chromatography including gel filtration. These and other methods may take advantage of some fusion partner or label of the PCyRPA polypeptide and/or α2-6-linked sialic acid (e.g. 2-6SLN). Assays may employ a variety of detection methods including but not limited to chromogenic, fluorescent, luminescent, or isotopic labels.

Compounds may be further tested in the animal models to identify those compounds having the most potent in vivo effects. These molecules may serve as “lead compounds” for the further development of pharmaceuticals by, for example, subjecting the compounds to sequential modifications, molecular modeling, and other routine procedures employed in rational drug design.

### 4. Pharmaceutical Compositions

While it is possible that, for use in therapy, a PCyRPA-interacting compound (i.e., PCyRPA ligand) may be administered in an undiluted form, it is preferable to present such a compound as a pharmaceutical composition.

A pharmaceutical composition may comprise a PCyRPA-interacting compound described herein and a pharmaceutically acceptable carrier. Carriers must be “acceptable” in the sense of being compatible with the other ingredients of the composition and not deleterious to the recipient thereof.

In accordance with the disclosure, a compound as described is administered under a therapeutic regime that is non-toxic to the subject.

The pharmaceutical compositions of the present disclosure or the compositions used in the methods of the present disclosure may be formulated and administered using methods known in the art. Techniques for formulation and administration may be found in, for example, Remington: The Science and Practice of Pharmacy, Loyd V. Allen, Jr (Ed), The Pharmaceutical Press, London, 22^{nd} Edition, September 2012.

The compositions of the disclosure may be formulated for administration by any route. In some embodiments the composition is formulated for oral administration. An oral composition may be in the form of tablets, capsules, powders, granules, or liquid preparations. In some embodiments the composition is formulated for topical administration. A topical composition may be in the form of a cream, lotion, ointment, gel, or shampoo. In some embodiments the composition is formulated for parenteral administration, for example by an intramuscular, intrathecal, intraperitoneal, intravaginal, intrauterine, intravesical or intravenous route.

As will be readily appreciated by those skilled in the art, the route of administration and the nature of the pharmaceutically acceptable carrier will depend on the nature of the condition and the mammal to be treated. It is believed that the choice of a particular carrier or delivery system, and route of administration could be readily determined by a person skilled in the art. In the preparation of any formulation containing the compound care should be taken to ensure that the activity of the compound is not destroyed in the process and that the compound is able to reach its site of action without being destroyed. In some circumstances it may be necessary to protect the compound by means known in the art, such as, for example, micro encapsulation or coating (such as the use of enteric coating). Similarly the route of administration chosen should be such that the compound reaches its site of action.

Those skilled in the art may readily determine appropriate formulations for the compounds of the present disclosure using conventional approaches. Identification of preferred pH ranges and suitable excipients, for example antioxidants, is routine in the art. Buffer systems are routinely used to provide pH values of a desired range and include carboxylic acid buffers for example acetate, citrate, lactate and succinate. A variety of antioxidants are available for such formulations including phenolic compounds such as BHT or vitamin E, and reducing agents such as methionine or sulfite.

The PCyRPA-interacting compounds described herein may be prepared in parenteral dosage forms, including those suitable for intravenous, intrathecal, and intracerebral or epidural delivery. The pharmaceutical forms suitable for injectable use include sterile injectable solutions or dispersions, and sterile powders for the extemporaneous preparation of sterile injectable solutions. They should be stable under the conditions of manufacture and storage and may be preserved against reduction or oxidation and the contaminating action of microorganisms such as bacteria or fungi.

The solvent or dispersion medium for the injectable solution or dispersion may contain any of the conventional solvent or carrier systems for the compound or proteinaceous molecule, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolarity, for example, sugars or sodium chloride. Preferably, the formulation for injection will be isotonic with blood. Prolonged absorption of the injectable compositions can be brought about by the use in the compositions of agents delaying absorption, for example, aluminum monostearate and gelatin. Pharmaceutical forms suitable for injectable use may be delivered by any appropriate route including intravenous, intramuscular, intracerebral, intrathecal, epidural injection, intravesicular administration or infusion.

Sterile injectable solutions are prepared by incorporating the active compound in the required amount in the appropriate solvent with various of the other ingredients such as those enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

Other pharmaceutical forms include oral and enteral formulations of a PCyRPA-interacting compound described herein, in which the active compound may be formulated with an inert diluent or with an edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal or sublingual tablets, troches, capsules, elixirs, suspensions, syrups, wafers, and the like. The amount of active compound in such therapeutically useful compositions is such that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavoring agent. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, and a sweetening agent, preservative, dye or flavoring.

Any component used in the preparation of any dosage unit form should be pharmaceutically pure and substantially non-toxic in the amounts employed.

The present disclosure also extends to any other forms suitable for administration, for example topical application such as creams, foams, washes, lotions, sprays, and gels; enteral formulations such as suppositories; or compositions suitable for inhalation or intranasal delivery, for example solutions, aerosols, dry powders, suspensions or emulsions. In specific embodiments, the compounds of the present disclosure are formulated for topical application to the skin or body cavity, such as shampoos, conditioners, scrubs, cosmetics, lotions, foams, creams, washes, gels, sprays, suppositories, pessaries, lotions, ointments, ovules, tampons, or aerosols.

Pharmaceutically acceptable vehicles and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It may be advantageous to formulate the compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutically acceptable vehicle. The specification for the novel dosage unit forms of the disclosure are dictated by and directly dependent on (a) the unique characteristics of the active material and the particular therapeutic effect to be achieved, and (b) the limitations inherent in the art of compounding active materials for the treatment of disease in living subjects having a diseased condition in which bodily health is impaired.

The principal active ingredient may be compounded for convenient and effective administration in therapeutically effective amounts with a suitable pharmaceutically acceptable vehicle in dosage unit form. A unit dosage form can, for example, contain the principal active compound in amounts ranging from 0.25 μg to about 1000 mg. Expressed in proportions, the active compound may be present in from about 0.25 μg to about 1000 mg/mL of carrier. In the case of compositions containing supplementary active ingredients, the dosages are determined by reference to the usual dose and manner of administration of the said ingredients.

In some embodiments, and dependent on the intended mode of administration, the PCyRPA-interacting compound-containing compositions will generally contain about 0.000001% to 90%, about 0.0001% to 50%, or about 0.01% to about 25%, by weight of PCyRPA-interacting compound, the remainder being suitable pharmaceutical carriers or diluents etc. In some embodiments in which the PCyRPA-interacting compound is Zinc Pyrithione, the composition suitably comprises Zinc Pyrithione in an amount of from 0.01 to 10%, preferably from about 0.2 to 2.0% (and all one decimal digit percentage unit dosages in between) by weight of the total composition. In some embodiments, the composition comprises about 0.25% by weight of Zinc Pyrithione. In some embodiments, the composition comprises about 1.0% by weight of Zinc Pyrithione. In some embodiments, the composition comprises about 1.5% by weight of Zinc Pyrithione. In some embodiments, the composition comprises about 2.0% by weight of Zinc Pyrithione. In some embodiments, the composition comprises about 10.0% by weight of Zinc Pyrithione. In some embodiments, the Zinc Pyrithione-containing composition is in a usage dosage form, each dosage containing from about 0.5 μg to about 10,000 mg (1 g) (and all integer microgram unit dosages in between), more usually about 1 mg to about 1000 mg (and all integer milligram unit dosages in between), of Zinc Pyrithione. In representative embodiments of these types, the Zinc Pyrithione-containing composition is suitably a topical composition such as a shampoo, conditioner, wash, scrub, cosmetic, lotion, foam, ointment, cream, tonic, gel, topical spray or hygiene product, suitably for use with water or administration to a wet surface. In other representative embodiments, the Zinc Pyrithione-containing composition is an oral composition (e.g., a tablet, capsule, powder, granule, or liquid preparation), or an enteral composition (e.g., suppository). In other representative embodiments, the Zinc Pyrithione-containing composition is a an injectable composition (e.g., for subcutaneous, intravenous, parenteral, intraperitoneal or intrathecal administration).

In some embodiments in which the PCyRPA-interacting compound is Zinc Pyrithione, the composition is suitably a topical composition such as a shampoo, conditioner, wash, scrub, cosmetic, lotion, foam, wash, ointment, cream, tonic, gel, topical spray or hygiene product, suitably for use with water or administration to a wet surface. The Zinc Pyrithione-containing composition may be formulated with an anionic surfactant e.g., an alkyl sulfate and/or ethoxylated alkyl sulfate surfactant. These anionic surfactants are suitably present at a level of from 2 to 16%, more preferably from 3 to 16% by weight of the composition. Preferred alkyl sulfates are C₈₋₁₈ alky sulfates, more preferably C₁₂₋₁₈ alkyl sulfates, preferably in the form of a salt with a solubilizing cation such as sodium, potassium, ammonium or substituted ammonium. Examples are sodium lauryl sulfate (SLS) or sodium dodecyl sulfate (SDS). Exemplary alkyl ether sulfates are those having the formula: RO(CH₂CH₂O) nSO-3M; wherein R is an alkyl or alkenyl having from 8 to 18 (preferably 12 to 18) carbon atoms; n is a number having an average value of greater than at least 0.5, preferably between 1 and 3, more preferably between 2 and 3; and M is a solubilizing cation such as sodium, potassium, ammonium or substituted ammonium. An example is sodium lauryl ether sulfate (SLES). A preferred ethoxylated alkyl sulfate anionic surfactant is sodium lauryl ether sulfate (SLES) having an average degree of ethoxylation of from 0.5 to 3, preferably 1 to 3. Zinc Pyrithione-containing compositions may optionally and preferably additionally comprises a betaine surfactant. In a preferred embodiment, the composition comprises from 0.1 to 10 wt. %, preferably from 0.5 to 8 wt. %, more preferably from 1 to 5 wt. % of a betaine surfactant, preferably an alkyl amidopropyl betaine, for example cocamidopropyl betaine.

Shampoo compositions of the present disclosure may comprise one or more further anionic cleansing surfactants which are cosmetically acceptable and suitable for topical application to the hair. Examples of further suitable anionic cleansing surfactants are the alkaryl sulfonates, alkyl succinates, alkyl sulphosuccinates, alkyl ether sulphosuccinates, N-alkyl sarcosinates, alkyl phosphates, alkyl ether phosphates, and alkyl ether carboxylic acids and salts thereof, especially their sodium, magnesium, ammonium and mono-, di- and triethanolamine salts. The alkyl and acyl groups generally contain from 8 to 18, preferably from 10 to 16 carbon atoms and may be unsaturated. The alkyl ether sulphosuccinates, alkyl ether phosphates and alkyl ether carboxylic acids and salts thereof may contain from 1 to 20 ethylene oxide or propylene oxide units per molecule. Typical anionic cleansing surfactants for use in shampoo compositions of the present disclosure include sodium oleyl succinate, ammonium lauryl sulphosuccinate, sodium lauryl ether sulphosuccinate, sodium dodecylbenzene sulfonate, triethanolamine dodecylbenzene sulfonate, lauryl ether carboxylic acid and sodium N-lauryl sarcosinate. Suitable preferred additional anionic cleansing surfactants are sodium lauryl ether sulphosuccinate(n)EO, (where n is from 1 to 3), lauryl ether carboxylic acid (n) EO (where n is from 10 to 20). Mixtures of any of the foregoing anionic cleansing surfactants may also be suitable. If added, the total amount of anionic cleansing surfactant in shampoo compositions of the present disclosure may generally range from 0.5 to 45 wt. %, preferably from 1.5 to 35 wt. %, more preferably from 5 to 20 wt. %, calculated by total weight anionic cleansing surfactant based on the total weight of the composition.

Representative hair conditioning compositions comprise conditioning surfactants selected from cationic surfactants, used singly or in admixture. Preferably, the cationic surfactants have the formula N+R1R2R3R4 wherein R1, R2, R3 and R4 are independently (C₁ to C₃₀) alkyl or benzyl. Preferably, one, two or three of R1, R2, R3 and R4 are independently (C₄ to C₃₀) alkyl and the other R1, R2, R3 and R4 group or groups are (C₁-C₆) alkyl or benzyl. More preferably, one or two of R1, R2, R3 and R4 are independently (C₆ to C₃₀) alkyl and the other R1, R2, R3 and R4 groups are (C₁-C₆) alkyl or benzyl groups. Optionally, the alkyl groups may comprise one or more ester (—OCO— or —COO—) and/or ether (—O—) linkages within the alkyl chain. Alkyl groups may optionally be substituted with one or more hydroxyl groups. Alkyl groups may be straight chain or branched and, for alkyl groups having 3 or more carbon atoms, cyclic. The alkyl groups may be saturated or may contain one or more carbon-carbon double bonds (e.g., oleyl). Alkyl groups are optionally ethoxylated on the alkyl chain with one or more ethyleneoxy groups. Suitable cationic surfactants for use in conditioner compositions according to the present disclosure include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (e.g., Arquad 2HT/75 from Akzo Nobel), cocotrimethylammonium chloride, PEG-2-oleammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in conditioners according to the present disclosure is cetyltrimethylammonium chloride, available commercially, for example as GENAMIN CTAC, ex Hoechst Celanese. Another particularly useful cationic surfactant for use in conditioners according to the present disclosure is behenyltrimethylammonium chloride, available commercially, for example as GENAMIN KDMP, ex Clariant. Yet another preferred cationic surfactant is stearamidopropyl dimethylamine. In preferred embodiments, the cationic surfactants for use in the conditioner compositions of the present disclosure are stearamidopropyl dimethylamine, behentrimonium chloride, or stearyl trimethyl ammonium chloride. In conditioners of the present disclosure, the level of cationic surfactant will generally range from 0.1 to 5%, preferably 0.5 to 2.5% by weight of the composition.

Hair conditioning compositions disclosed herein preferably may also additionally comprise a fatty alcohol. The combined use of fatty alcohols and cationic surfactants in conditioning compositions is believed to be especially advantageous, because this leads to the formation of a lamellar phase, in which the cationic surfactant is dispersed. Representative fatty alcohols comprise from 8 to 22 carbon atoms, more preferably 16 to 22. Fatty alcohols are typically compounds containing straight chain alkyl groups. Examples of suitable fatty alcohols include cetyl alcohol, stearyl alcohol and mixtures thereof. The use of these materials is also advantageous in that they contribute to the overall conditioning properties of compositions of the present disclosure. The level of fatty alcohol in conditioners of the present disclosure will generally range from 0.5 to 10%, preferably from 0.1 to 8%, more preferably from 0.2 to 7%, most preferably from 0.3 to 6% by weight of the composition. The weight ratio of cationic surfactant to fatty alcohol is suitably from 1:1 to 1:10, more preferably from 1:1.5 to 1:8, optimally from 1:2 to 1:5.

In some embodiments in which the PCyRPA-interacting compound is Ivermectin, the composition is in a unit dosage form, each dosage containing from about 20 to about 1,000 mg (1 g), more usually about 20 mg to about 500 mg (and all integer milligram unit dosages in between), of Ivermectin. In some embodiments, each dosage contains about 20 mg of the active ingredient. In some embodiments, each dosage contains about 30 mg of Ivermectin. In some embodiments, each dosage contains about 40 mg of Ivermectin. In some embodiments, each dosage contains about 50 mg of Ivermectin. In some embodiments, each dosage contains about 60 mg of Ivermectin. In some embodiments, each dosage contains about 70 mg of Ivermectin. In some embodiments, each dosage contains about 80 mg of Ivermectin. In some embodiments, each dosage contains about 90 mg of Ivermectin. In some embodiments, each dosage contains about 100 mg of Ivermectin. In some embodiments, each dosage contains about 110 mg of Ivermectin. In some embodiments, each dosage contains about 120 mg of Ivermectin. Suitably, the Ivermectin-containing composition is administered to a patient at a dosage of, for example, from about 0.3 mg/kg to about 7.5 mg/kg (and all one decimal digit milligram unit dosages in between). The dosage may be for any suitable period, including for example, daily, three times a week, twice a week, weekly, fortnightly, three times a month, three monthly, four monthly, five monthly, half yearly or yearly. In other embodiments, the Ivermectin-containing composition is an oral composition (e.g., tablet, capsule, powder, granule, or liquid preparation) or enteral composition (e.g., suppository). In other embodiments, the Ivermectin-containing composition is a an injectable composition (e.g., for subcutaneous, intravenous, parenteral, intraperitoneal or intrathecal administration). In still other embodiments, the Ivermectin-containing composition is a topical composition (e.g., a shampoo, conditioner, wash, scrub, cosmetic, lotion, foam, ointment, cream, tonic, gel, topical spray or hygiene product, suitably for use with water or administration to a wet surface).

In some embodiments in which the PCyRPA-interacting compound is RIVANOL, the composition suitably comprises RIVANOL in an amount of from 0.01 to 10%, preferably from about 0.05 to 5.0% (and all one decimal digit percentage unit dosages in between) by weight of the total composition. In some embodiments, the composition comprises about 0.1% by weight of RIVANOL. In some embodiments, the composition comprises about 0.2% by weight RIVANOL. In some embodiments, the composition comprises about 1.0% by weight of RIVANOL. In some embodiments, the composition comprises about 5.0% by weight of RIVANOL. In representative embodiments of this type, the RIVANOL-containing composition is suitably an oral composition e.g., tablet, capsule, powder, granule, or liquid preparation), an enteral composition (e.g., suppository) or a topical composition (e.g., shampoos, conditioners, scrubs, cosmetics, lotions, foams, creams, washes, gels, sprays, suppositories, pessaries, lotions, ointments, ovules, tampons, or aerosols). In other embodiments, the RIVANOL-containing composition is an injectable composition (e.g., for subcutaneous, intravenous, parenteral, intraperitoneal or intrathecal administration). In still other embodiments, the RIVANOL-containing composition is a topical composition (e.g., a shampoo, conditioner, wash, scrub, cosmetic, lotion, foam, ointment, cream, tonic, gel, topical spray or hygiene product, suitably for use with water or administration to a wet surface).

In any event, the dosage of a PCyRPA-interacting compound disclosed herein can depend on a variety of factors, such as mode of administration, the species of the affected subject, age and/or individual condition, and can be easily determined by a person of skill in the art using standard protocols.

In any of the aspects and embodiments disclosed herein, the PCyRPA-interacting compound may be administered at a daily dose of between about 0.1 mg/kg and 400 mg/kg (and all one tenth integer mg/kg units in between), or between about 0.2 mg/kg and 20 mg/kg (and all one tenth integer mg/kg units in between), or between about 0.5 mg/kg and 15 mg/kg (and all one tenth integer mg/kg units in between), or between about 1 mg/kg and 10 mg/kg (and all integer mg/kg units in between). The daily dose is suitably administered in a single dose or in two doses.

In any of the aspects and embodiments disclosed herein, the PCyRPA-interacting compound may be administered at a weekly dose of between about 0.1 mg/kg and 400 mg/kg (and all one tenth integer mg/kg units in between), or between about 0.2 mg/kg and 20 mg/kg (and all one tenth integer mg/kg units in between), or between about 0.5 mg/kg and 15 mg/kg (and all one tenth integer mg/kg units in between), or between about 1 mg/kg and 10 mg/kg (and all integer mg/kg units in between).

In any of the aspects and embodiments disclosed herein, the PCyRPA-interacting compound is administered to the subject over a period of about 1 day, or over a period of about 2 days, or over a period of about 3 days, or over a period of about 4 days, or over a period of about 5 days, or over a period of about 6 days, over a period of about 1 week, or over a period of about 2 weeks, or over a period of about 3 weeks, or over a period of about 4 weeks, or over a period of about 5 weeks, or over a period of about 6 weeks, or over a period of about 2 months, or over a period of about 3 months, or over a period of about 4 months, or over a period of about 5 months, or over a period of about 6 months, or over a period of about 7 months, or over a period of about 8 months, or over a period of about 9 months, or over a period of about 10 months, or over a period of about 11 months, or over a period of about 1 year.

In any event, dosages can be empirically determined considering the type and stage of disease diagnosed in a particular patient. The dose administered to a patient, in the context of the present disclosure should be sufficient to effect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, or via a patch or pump.

A PCyRPA-interacting compound as described herein, may be the sole active ingredient administered to the subject. However, it will be appreciated that the compound may be administered with an ancillary agent (e.g., a further therapeutic agent such as an antimalarial agent or an adjunctive antimalarial therapeutic agent). Accordingly, the present disclosure contemplates administering a PCyRPA-interacting compound described herein with one or more further ancillary agents in combination. The combination may allow for concurrent administration (e.g., separate, sequential or simultaneous administration) of the compound with the other active ingredient(s). The combination may be provided in the form of a pharmaceutical composition. Administration with one or more other active ingredients is within the scope of the disclosure.

In specific embodiments, a PCyRPA-interacting compound described herein is administered concurrently with an antimalarial agent (also referred to herein as an ancillary antimalarial agent), which includes without limitation compounds that act directly on a Plasmodium pathogen and/or kill or inhibit the growth, replication or persistence of a Plasmodium pathogen, particularly P. falciparum. Such antimalarial agents include but are not limited to quinolines (e.g., quinine, chloroquine, amodiaquine, mefloquine, primaquine, tafenoquine), peroxide antimalarials (e.g., artemisinin, artemether, artesunate), pyrimethamine-sulfadoxine antimalarials (e.g., Fansidar), hydroxynaphtoquinones (e.g., atovaquone), acroline-type antimalarials (e.g., pyronaridine) and other antiprotozoal agents like ethylstibamine, hydroxystilbamidine, pentamidine, stilbamidine, quinapyramine, puromycine, propamidine, nifurtimox, melarsoprol, nimorazole, nifuroxime, aminitrozole and the like.

Alternatively or in addition, a PCyRPA-interacting compound described herein may be administered concurrently with an adjunctive therapy for improving efficacy, or reducing malarial disease-associated complications. Illustrative examples of adjunctive therapies for treatment of malaria include corticosteroids such as dexamethasone, anti-malarial immunoglobulin, curdlan sulfate, anti-tumor necrosis factor (TNF) antibodies, oral activated charcoal, and peroxisome proliferator-activated receptor-γ (PPAR-γ) agonists.

It is conceivable that more than one administration of either the PCyRPA-interacting compound or ancillary agent will be desired. Various combinations may be employed, where the PCyRPA-interacting compound is “A” and the ancillary agent is “B”, as exemplified below:

A/B/A B/A/B B/B/A A/A/B B/A/A A/B/B B/B/B/A B/B/A/B A/A/B/B
A/B/A/B A/B/B/A B/B/A/A B/A/B/A B/A/A/B B/B/B/A A/A/A/B
B/A/A/A A/B/A/A A/A/B/A A/B/B/B B/A/B/B B/B/A/B.

Pharmaceutical compositions of the present disclosure may be provided in a kit. The kit may comprise additional components to assist in performing the methods of the present disclosure such as, for example, administration device(s), buffer(s), and/or diluent(s). The kits may include containers for housing the various components and instructions for using the kit components in the methods of the present disclosure.

### EMBODIMENTS OF THE DISCLOSURE

1. A method for inhibiting interaction of a Plasmodium cysteine-rich protective antigen (CyRPA) with a cell that is capable of being infected by a Plasmodium pathogen, the method comprising, consisting or consisting essentially of contacting the CyRPA with a ligand of the CyRPA which when bound to the CyRPA inhibits interaction of the CyRPA with the cell.

2. A method for treating or inhibiting the development of a Plasmodium pathogen infection in a subject, the method comprising, consisting or consisting essentially of administering to the subject an effective amount of a ligand of a cysteine-rich protective antigen (CyRPA) expressed by the Plasmodium pathogen which ligand, when bound to the CyRPA, inhibits interaction of the CyRPA with a cell that is capable of being infected by the Plasmodium pathogen, to thereby treat or inhibit the development of the Plasmodium pathogen infection in the subject.

3. The method of embodiment 1 or embodiment 2, wherein the CyRPA interacts with a glycan expressed by the cell when the CyRPA is not bound to the ligand and wherein when the CyRPA is bound to the ligand, the ligand inhibits interaction of the CyRPA with the glycan.

4. The method of embodiment 3, wherein the glycan comprises an α2-6-linked sialic acid.

5. The method of embodiment 4, wherein the glycan comprises α2-6-Neu5Ac.

6. The method of embodiment 4 or embodiment 5, wherein the glycan comprises α2-6-sialyllactosamine (Neu5Ac-α-(2-6)-Gal-β-(1-4)-GlcNAc).

7. The method of any one of embodiments 1 to 6, wherein the interaction is one or both of binding of the Plasmodium pathogen to the cell and entry of the Plasmodium pathogen into the cell.

8. The method of any one of embodiments 1 to 7, wherein the cell is an erythrocyte or hepatocyte.

9. The method of any one of embodiments 1 to 7, wherein the cell is an erythrocyte.

10. The method of any one of embodiments 1 to 9, wherein the ligand is an effective amount that inhibits entry of the Plasmodium pathogen into the cell.

11. The method of any one of embodiments 1 to 10, wherein the ligand is in an effective amount that inhibits invasion of the cell by the Plasmodium pathogen.

12. The method of any one of embodiments 1 to 11, wherein the ligand is in an effective amount that inhibits replication of the Plasmodium pathogen in the cell.

13. The method of any one of embodiments 2 to 12, wherein the ligand is in an effective amount that inhibits spreading of the Plasmodium pathogen within the subject.

14. The method of any one of embodiments 1 to 13, wherein the CyRPA ligand is formulated for oral delivery.

15. The method of any one of embodiments 1 to 13, wherein the CyRPA ligand is formulated for enteral delivery.

16. The method of any one of embodiments 1 to 13, wherein the CyRPA ligand is formulated for systemic delivery.

17. The method of any one of embodiments 1 to 13, wherein the CyRPA ligand is formulated for topical delivery.

18. The method of any one of embodiments 1 to 13, wherein the CyRPA ligand is formulated for injection.

19. The method of any one of embodiments 2 to 18, wherein the subject is infected with the Plasmodium pathogen or is identified as being infected with the Plasmodium pathogen.

20. The method of any one of embodiments 2 to 18, wherein the subject is not infected with the Plasmodium pathogen.

21. The method of embodiment 20, wherein the uninfected subject is planning to visit or is visiting a malaria endemic area.

22. The method of any one of embodiments 1 to 21, wherein the Plasmodium pathogen is a drug-resistant Plasmodium pathogen.

23. The method of any one of embodiments 1 to 22, wherein the Plasmodium pathogen is a multidrug-resistant Plasmodium pathogen.

24. The method of any one of embodiments 2 to 22, wherein the Plasmodium pathogen is a multidrug-resistant Plasmodium pathogen and the subject is administered the CyRPA-interacting ligand when the subject is identified as being infected with a drug-resistant Plasmodium pathogen.

25. The method of any one of embodiments 2 to 22, wherein the Plasmodium pathogen is a multidrug-resistant Plasmodium pathogen and the subject is administered the CyRPA-interacting ligand after the subject has been administered at least one antimalarial drug to which the Plasmodium pathogen is resistant.

26. The method of embodiment 25, wherein the antimalarial drug is a drug that acts directly on a drug sensitive Plasmodium pathogen by killing the drug sensitive Plasmodium pathogen or inhibiting its growth, survival and/or viability.

27. The method of any one of embodiments 2 to 26, wherein the CyRPA ligand is administered concurrently with an ancillary agent.

28. The method of embodiment 27, wherein the ancillary agent is an antimalarial agent.

29. The method of embodiment 28, wherein the antimalarial agent acts directly on a Plasmodium pathogen and/or kills or inhibits the growth, replication or persistence of a Plasmodium pathogen.

30. The method of embodiment 28 or embodiment 29, wherein the antimalarial agent is selected from quinolines (e.g., quinine, chloroquine, amodiaquine, mefloquine, primaquine, tafenoquine), peroxide antimalarials (e.g., artemisinin, artemether, artesunate), pyrimethamine-sulfadoxine antimalarials (e.g., Fansidar), hydroxynaphtoquinones (e.g., atovaquone), acroline-type antimalarials (e.g., pyronaridine) and other antiprotozoal agents like ethylstibamine, hydroxystilbamidine, pentamidine, stilbamidine, quinapyramine, puromycine, propamidine, nifurtimox, melarsoprol, nimorazole, nifuroxime and aminitrozole.

31. The method of embodiment 27, wherein the ancillary agent is an adjunctive therapy for improving efficacy, or reducing one or more malarial disease-associated complications.

32. The method of embodiment 31, wherein the adjunctive therapy is selected from corticosteroids such as dexamethasone, anti-malarial immunoglobulin, curdlan sulfate, anti-tumor necrosis factor (TNF) antibodies, oral activated charcoal, and peroxisome proliferator-activated receptor-γ (PPAR-γ) agonists.

33. A composition for use in therapy or prophylaxis of a Plasmodium pathogen infection in a subject, the composition comprising, consisting or consisting essentially of a ligand of a P. falciparum CyRPA which, when bound to the CyRPA, inhibits interaction of the CyRPA with an α2-6-linked sialic acid of an α2-6-linked sialic acid-expressing cell, and optionally a pharmaceutically acceptable carrier.

34. A kit for use in treating a Plasmodium pathogen infection in a subject, the kit comprising: a ligand of a Plasmodium pathogen CyRPA which, when bound to the CyRPA, inhibits interaction of the CyRPA with an α2-6-linked sialic acid of an α2-6-linked sialic acid-expressing cell, and optionally instructional material for performing the treatment.

35. The kit for use according to embodiment 28, further comprising an ancillary agent useful for the treatment of the Plasmodium pathogen infection.

36. The kit of embodiment 35, wherein the ancillary agent is an antimalarial agent.

37. The kit of embodiment 36, wherein the antimalarial agent acts directly on a Plasmodium pathogen and/or kills or inhibits the growth, replication or persistence of a Plasmodium pathogen.

38. The kit of embodiment 36 or embodiment 37, wherein the antimalarial agent is selected from quinolines (e.g., quinine, chloroquine, amodiaquine, mefloquine, primaquine, tafenoquine), peroxide antimalarials (e.g., artemisinin, artemether, artesunate), pyrimethamine-sulfadoxine antimalarials (e.g., Fansidar), hydroxynaphtoquinones (e.g., atovaquone), acroline-type antimalarials (e.g., pyronaridine) and other antiprotozoal agents like ethylstibamine, hydroxystilbamidine, pentamidine, stilbamidine, quinapyramine, puromycine, propamidine, nifurtimox, melarsoprol, nimorazole, nifuroxime and aminitrozole.

39. The kit of embodiment 35, wherein the ancillary agent is an adjunctive therapy for improving efficacy, or reducing one or more malarial disease-associated complications.

40. The kit of embodiment 39, wherein the adjunctive therapy is selected from corticosteroids such as dexamethasone, anti-malarial immunoglobulin, curdlan sulfate, anti-tumor necrosis factor (TNF) antibodies, oral activated charcoal, and peroxisome proliferator-activated receptor-γ (PPAR-γ) agonists.

41. A method for identifying an agent that inhibits interaction of a Plasmodium pathogen with an α2-6-linked sialic acid-expressing cell, or that inhibits entry of a Plasmodium pathogen into an α2-6-linked sialic acid-expressing cell, or that inhibits invasion of an α2-6-linked sialic acid-expressing cell by a Plasmodium pathogen, or that is useful for treating or inhibiting the development of a Plasmodium pathogen infection, the method comprising: contacting a Plasmodium pathogen CyRPA polypeptide with a candidate agent; detecting binding of the candidate agent to the CyRPA polypeptide, and determining whether the candidate agent inhibits binding of the CyRPA polypeptide to an α2-6-linked sialic acid, wherein presence of the inhibition indicates that the candidate agent is an agent that inhibits interaction of a Plasmodium pathogen with an α2-6-linked sialic acid-expressing cell, or that inhibits entry of a Plasmodium pathogen into an α2-6-linked sialic acid-expressing cell, or that inhibits invasion of an α2-6-linked sialic acid-expressing cell by a Plasmodium pathogen, or that is useful for treating or inhibiting the development of a Plasmodium pathogen infection.

42. The method of any one of embodiments 1 to 32, or the composition of embodiment 33, or the kit for use of any one of embodiments 34-40, wherein the CyRPA ligand is selected from the compounds listed in TABLE 1.

TABLE 1

| | |
|---|---|
| 1. A compound of formula (I): | |
| 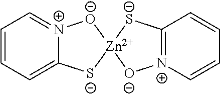 | (I) |
| (Zinc Pyrithione) | |
| 2. A compound of formula (II): | |
| 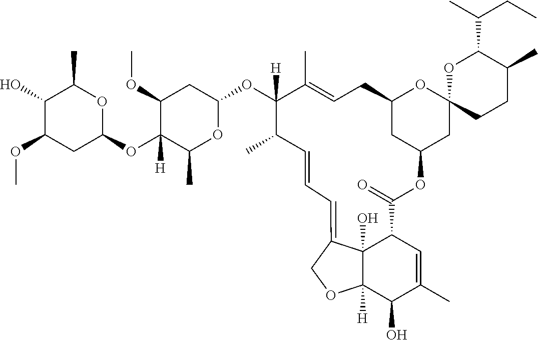 [SMILES: CCC(C)[C@H]1O[C@]2(CC[C@@H]1C)C[C@@H]1C[C@@H](C/C=C(\C)[C@@H](O[C@H]3C[C@H](OC)[C@@H](O[C@H]4C[C@@H](OC)[C@H](O)[C@@H](C)O4)[C@H](C)O3)[C@@H](C)/C=C/C=C3\CO[C@@H]4[C@H](O)C(C)=C[C@@H](C(=O)O1)[C@]34O)O2] | (II) |
| (Ivermectin) | |
| 3. A compound of formula (III): | |
| 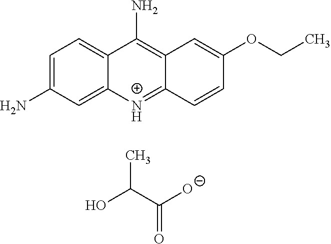 | (III) |
| (RIVANOL) | |

43. A composition for use in therapy or prophylaxis of a Plasmodium pathogen infection in a subject, the composition comprising, consisting or consisting essentially of a compound of formula (I):

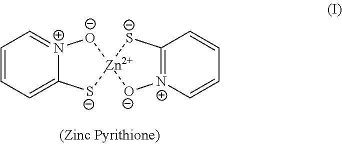

and optionally a pharmaceutically acceptable carrier.

44. The composition for use of embodiment 43, wherein the composition is formulated for topical administration.

45. The composition for use of embodiment 44, wherein the composition is a shampoo, conditioner, wash, scrub, cosmetic, lotion, foam, ointment, cream, tonic, gel, topical spray or hygiene product, suitably for use with water or administration to a wet surface.

46. The composition for use of embodiment 43, wherein the composition is formulated for oral administration.

47. The composition for use of embodiment 46, wherein the composition is a tablet, capsule, powder, granule, or liquid preparation.

48. The composition for use of embodiment 43, wherein the composition is formulated for enteral administration.

49. The composition for use of embodiment 48, wherein the composition is a suppository.

50. The composition for use of embodiment 43, wherein the composition is an injectable composition.

51. The composition for use of embodiment 50, wherein the injectable composition is for subcutaneous, intravenous, parenteral, intraperitoneal or intrathecal administration.

52. The composition for use of any one of embodiments 43 to 51, wherein the Zinc Pyrithione is present in an amount of from 0.01 to 10%, preferably from about 0.2 to 2.0% (and all one decimal digit percentage unit dosages in between) by weight of the total composition.

53. The composition for use of any one of embodiments 43 to 52, wherein the Zinc Pyrithione-containing composition is in a usage dosage form, each dosage containing from about 0.5 μg to about 10,000 mg (1 g) (and all integer microgram unit dosages in between), more usually about 1 mg to about 1000 mg (and all integer milligram unit dosages in between), of Zinc Pyrithione.

54. A composition for use in therapy of a Plasmodium pathogen infection in a subject, the composition comprising, consisting or consisting essentially of a compound of formula (II):

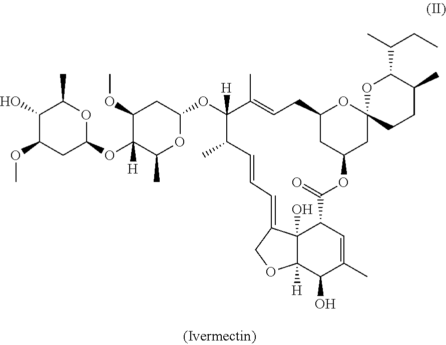 [SMILES: CCC(C)[C@H]1O[C@]2(CC[C@@H]1C)C[C@@H]1C[C@@H](C/C=C(\C)[C@@H](O[C@H]3C[C@H](OC)[C@@H](O[C@H]4C[C@@H](OC)[C@H](O)[C@@H](C)O4)[C@H](C)O3)[C@@H](C)/C=C/C=C3\CO[C@@H]4[C@H](O)C(C)=C[C@@H](C(=O)O1)[C@]34O)O2]

and optionally a pharmaceutically acceptable carrier.

55. The composition for use of embodiment 54, wherein the composition is formulated for oral administration.

56. The composition for use of embodiment 55, wherein the composition is a tablet, capsule, powder, granule, or liquid preparation.

57. The composition for use of embodiment 54, wherein the composition is formulated for enteral administration.

58. The composition for use of embodiment 57, wherein the composition is a suppository.

59. The composition for use of embodiment 54, wherein the composition is an injectable composition.

60. The composition for use of embodiment 59, wherein the injectable composition is for subcutaneous, intravenous, parenteral, intraperitoneal or intrathecal administration.

61. The composition for use of embodiment 54, wherein the composition is a topical composition.

62. The composition for use of embodiment 61, wherein topical composition is a shampoo, conditioner, wash, scrub, cosmetic, lotion, foam, ointment, cream, tonic, gel, topical spray or hygiene product, suitably for use with water or administration to a wet surface.

63. The composition for use of any one of embodiments 54 to 62, wherein the composition is in a unit dosage form, each dosage containing from about 20 to about 1,000 mg (1 g), more usually about 20 mg to about 500 mg (and all integer milligram unit dosages in between), of Ivermectin.

64. A composition for use in therapy or prophylaxis of a Plasmodium pathogen infection in a subject, the composition comprising, consisting or consisting essentially of a compound of formula (III):

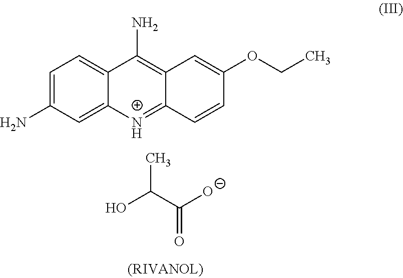

and optionally a pharmaceutically acceptable carrier.

65. The composition for use of embodiment 64, wherein the composition is formulated for oral administration.

66. The composition for use of embodiment 65, wherein the composition is a tablet, capsule, powder, granule, or liquid preparation.

67. The composition for use of embodiment 64, wherein the composition is formulated for enteral administration.

68. The composition for use of embodiment 67, wherein the composition is a suppository.

69. The composition for use of embodiment 64, wherein the composition is an injectable composition.

70. The composition for use of embodiment 69, wherein the injectable composition is for subcutaneous, intravenous, parenteral, intraperitoneal or intrathecal administration.

71. The composition for use of embodiment 64, wherein the composition is a topical composition.

72. The composition for use of embodiment 71, wherein topical composition is a shampoo, conditioner, wash, scrub, cosmetic, lotion, foam, ointment, cream, tonic, gel, topical spray or hygiene product, suitably for use with water or administration to a wet surface.

73. The composition for use of any one of embodiments 64 to 72, wherein comprises RIVANOL in an amount of from 0.01 to 10%, preferably from about 0.05 to 5.0% (and all one decimal digit percentage unit dosages in between) by weight of the total composition.

74. A method for treating or inhibiting the development of a Plasmodium pathogen infection in a subject the method comprising, consisting or consisting essentially of administering to the subject an effective amount of a compound of formula (I):

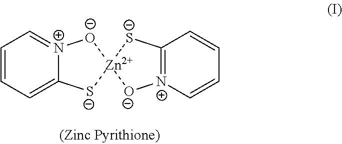

to thereby treat or inhibit the development of the Plasmodium pathogen infection in the subject.

75. The method of embodiment 74, comprising topically administering the Zinc Pyrithione to the subject.

76. The method of embodiment 75, wherein the Zinc Pyrithione is administered in the form of a shampoo, conditioner, wash, scrub, cosmetic, lotion, foam, ointment, cream, tonic, gel, topical spray or hygiene product.

77. The method of embodiment 74, comprising orally administering the Zinc Pyrithione to the subject.

78. The method of embodiment 77, wherein the Zinc Pyrithione is administered in the form of a tablet, capsule, powder, granule, or liquid preparation.

79. The method of embodiment 74, comprising enterally administering the Zinc Pyrithione to the subject.

80. The method of embodiment 79, wherein the Zinc Pyrithione is administered in the form of a suppository.

81. The method of embodiment 74, comprising systemically administering the Zinc Pyrithione to the subject by injection.

82. The method of embodiment 81, comprising administering the Zinc Pyrithione through the subcutaneous, intravenous, parenteral, intraperitoneal or intrathecal route.

83. A method for treating a Plasmodium pathogen infection in a subject the method comprising, consisting or consisting essentially of administering to the subject an effective amount of a compound of formula (II):

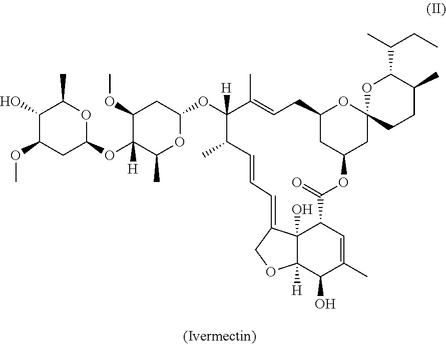 [SMILES: CCC(C)[C@H]1O[C@]2(CC[C@@H]1C)C[C@@H]1C[C@@H](C/C=C(\C)[C@@H](O[C@H]3C[C@H](OC)[C@@H](O[C@H]4C[C@@H](OC)[C@H](O)[C@@H](C)O4)[C@H](C)O3)[C@@H](C)/C=C/C=C3\CO[C@@H]4[C@H](O)C(C)=C[C@@H](C(=O)O1)[C@]34O)O2]

84. The method of embodiment 83, wherein comprising orally administering the Ivermectin to the subject.

85. The method of embodiment 84, wherein the Ivermectin is administered in the form of a tablet, capsule, powder, granule, or liquid preparation.

86. The method of embodiment 83, comprising enterally administering the Ivermectin to the subject.

87. The method of embodiment 86, wherein the Ivermectin is administered in the form of a suppository.

88. The method of embodiment 83, comprising systemically administering the Ivermectin to the subject by injection.

89. The method of embodiment 88, comprising administering the Ivermectin through the subcutaneous, intravenous, parenteral, intraperitoneal or intrathecal route.

90. The method of embodiment 83, comprising topically administering the Ivermectin to the subject.

91. The method of embodiment 90, wherein the Ivermectin is administered in the form of a shampoo, conditioner, wash, scrub, cosmetic, lotion, foam, ointment, cream, tonic, gel, topical spray or hygiene product.

92. A method for treating or inhibiting the development of a Plasmodium pathogen infection in a subject the method comprising, consisting or consisting essentially of administering to the subject an effective amount of a compound of formula (III):

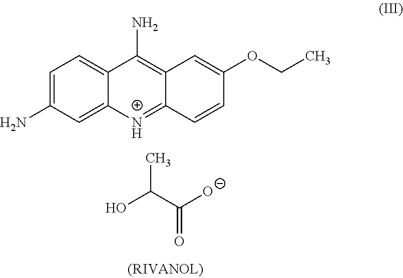

93. The method of embodiment 92, wherein comprising orally administering the RIVANOL to the subject.

94. The method of embodiment 93, wherein the RIVANOL is administered in the form of a tablet, capsule, powder, granule, or liquid preparation.

95. The method of embodiment 92, comprising enterally administering the RIVANOL to the subject.

96. The method of embodiment 95, wherein the RIVANOL is administered in the form of a suppository.

97. The method of embodiment 92, comprising systemically administering the RIVANOL to the subject by injection.

98. The method of embodiment 97, comprising administering the RIVANOL through the subcutaneous, intravenous, parenteral, intraperitoneal or intrathecal route.

99. The method of embodiment 92, comprising topically administering the RIVANOL to the subject.

100. The method of embodiment 99, wherein the RIVANOL is administered in the form of a shampoo, conditioner, wash, scrub, cosmetic, lotion, foam, ointment, cream, tonic, gel, topical spray or hygiene product.

101. The method of any one of embodiments 74 to 100, wherein the subject is infected with the Plasmodium pathogen or is identified as being infected with the Plasmodium pathogen.

102. The method of any one of embodiments 74 to 100, wherein the subject is not infected with the Plasmodium pathogen.

103. The method of embodiment 102, wherein the uninfected subject is planning to visit or is visiting a malaria endemic area.

104. The method of any one of embodiments 74 to 103, wherein the Plasmodium pathogen is a drug-resistant Plasmodium pathogen.

105. The method of any one of embodiments 74 to 104, wherein the Plasmodium pathogen is a multidrug-resistant Plasmodium pathogen.

106. The method of any one of embodiments 74 to 105, comprising identifying the subject as being infected with a drug-resistant Plasmodium pathogen.

107. The method of any one of embodiments 74 to 106, comprising administering the compound after the subject has been administered at least one antimalarial drug to which the Plasmodium pathogen is resistant.

108. The method of embodiment 107, wherein the antimalarial drug is a drug that acts directly on a drug sensitive Plasmodium pathogen by killing the drug sensitive Plasmodium pathogen or inhibiting its growth, survival and/or viability.

109. The method of any one of embodiments 74 to 108, wherein the CyRPA ligand is administered concurrently with an ancillary agent.

110. The method of embodiment 109, wherein the ancillary agent is an antimalarial agent.

111. The method of embodiment 110, wherein the antimalarial agent acts directly on a Plasmodium pathogen and/or kills or inhibits the growth, replication or persistence of a Plasmodium pathogen.

112. The method of embodiment 110 or embodiment 111, wherein the antimalarial agent is selected from quinolines (e.g., quinine, chloroquine, amodiaquine, mefloquine, primaquine, tafenoquine), peroxide antimalarials (e.g., artemisinin, artemether, artesunate), pyrimethamine-sulfadoxine antimalarials (e.g., Fansidar), hydroxynaphtoquinones (e.g., atovaquone), acroline-type antimalarials (e.g., pyronaridine) and other antiprotozoal agents like ethylstibamine, hydroxystilbamidine, pentamidine, stilbamidine, quinapyramine, puromycine, propamidine, nifurtimox, melarsoprol, nimorazole, nifuroxime and aminitrozole.

113. The method of embodiment 109, wherein the ancillary agent is an adjunctive therapy for improving efficacy, or reducing one or more malarial disease-associated complications.

114. The method of embodiment 113, wherein the adjunctive therapy is selected from corticosteroids such as dexamethasone, anti-malarial immunoglobulin, curdlan sulfate, anti-tumor necrosis factor (TNF) antibodies, oral activated charcoal, and peroxisome proliferator-activated receptor-γ (PPAR-γ) agonists.

115. The method, composition or kit of any preceding embodiment, wherein the Plasmodium pathogen is a Plasmodium pathogen that infects a primate.

116. The method, composition or kit of embodiment 115, wherein the primate is human.

117. The method, composition or kit of any preceding claim, wherein the Plasmodium pathogen is selected from P. falciparum, P. malariae, P. vivax, P. ovale, P. knowlesi, P. cynomolgi, and P. reichenowi.

118. The method, composition or kit any preceding claim, wherein the Plasmodium pathogen is P. falciparum or P. vivax.

In order that the disclosure may be readily understood and put into practical effect, particular preferred embodiments will now be described by way of the following non-limiting examples.

### EXAMPLES

### Example 1

Plasmodium Falciparum CyRPA Invasion Complex Protein is a Sialic Acid Lectin that Preferentially Binds 2,6 Linked Sialic Acid Structures.

Plasmodium falciparum is a human-adapted parasite that causes malaria. Cysteine-Rich Protective Antigen (PfCyRPA) is a P. falciparum invasion complex protein essential for erythrocyte invasion. PfCyRPA plays an essential role in erythrocyte invasion (Dreyer, Matile et al. 2012, supra). The tridimensional structure of PfCyRPA (Chen, Xu et al. 2017, supra; Favuzza, Guffart et al. 2017, supra) revealed that it adopts a six-bladed β-propeller fold and that its overall structure resembles the catalytic domain of neuraminidases. While PfCyRPA contains an Asp-box as sialidase signature motif, it lacks key residues necessary for catalysis, providing a structural correlate of the absence of enzymatic activity (Dreyer, Matile et al. 2012, supra).

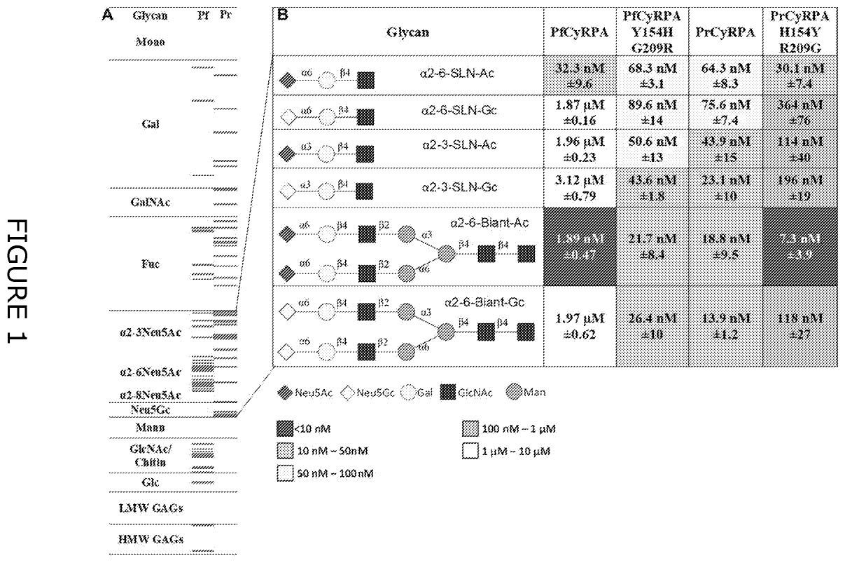

The present inventors hypothesized that PfCyRPA has evolved from a genuine sialidase, lost its enzymatic activity, but retained saccharide binding activity to meet other functionalities. To test this hypothesis, they conducted a glycan microarray-based binding analysis. Recombinantly expressed PfCyRPA bound to 43/402 glycans present on the array with strong preference for glycans terminating with α2-6-linked sialic acid (See FIGS. 1A and B). The highest affinity ligand identified by Surface Plasmon Resonance (SPR) analysis was α2-6-sialyllactosamine (α2-6-SLN-Ac, Neu5Ac-α-(2-6)-Gal-β-(1-4)-GlcNAc; FIG. 1B).

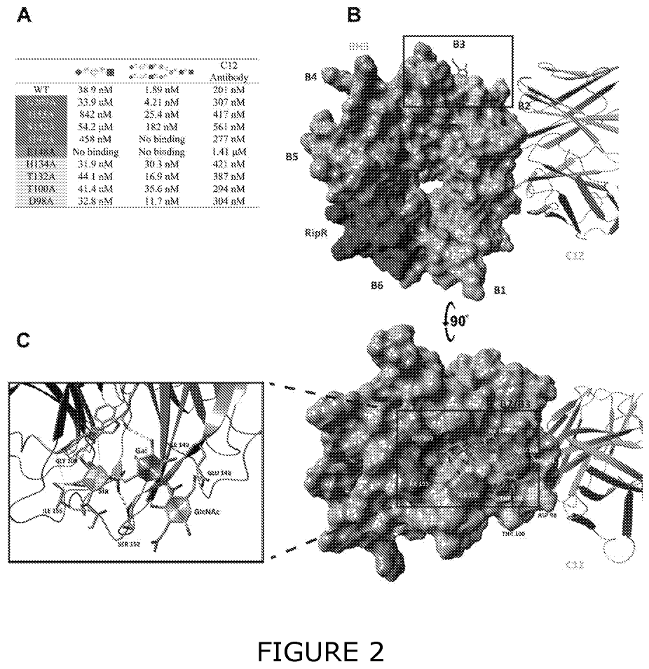

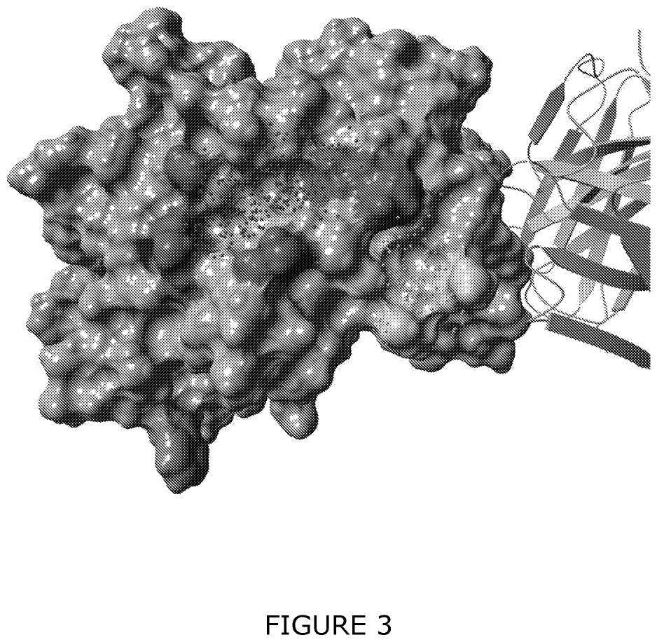

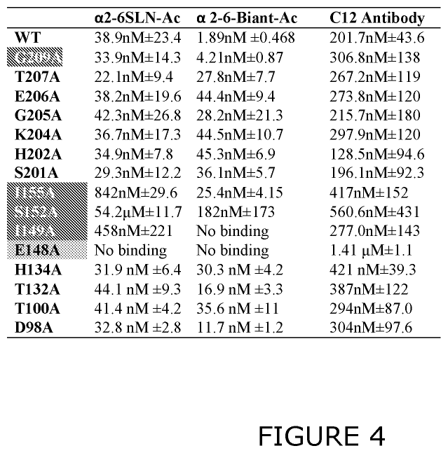

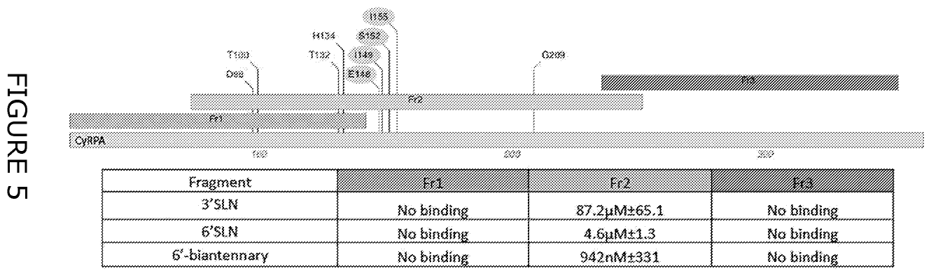

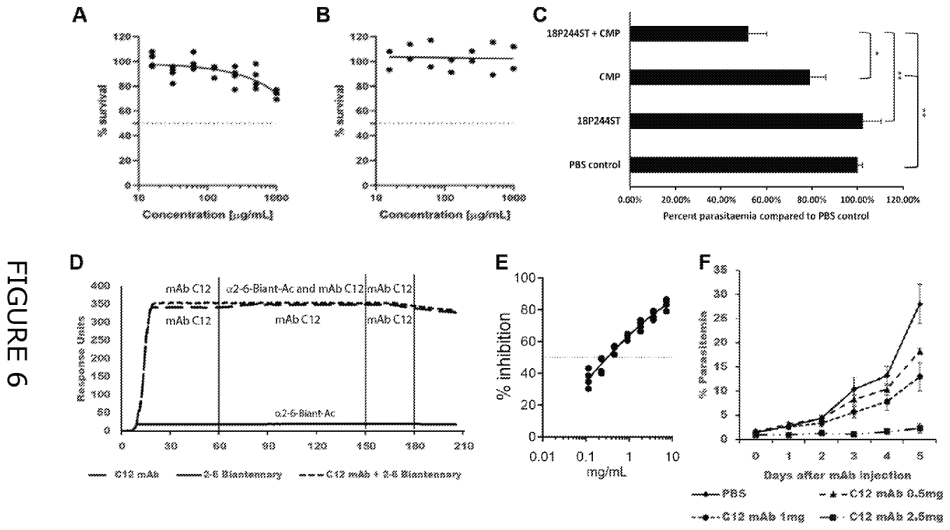

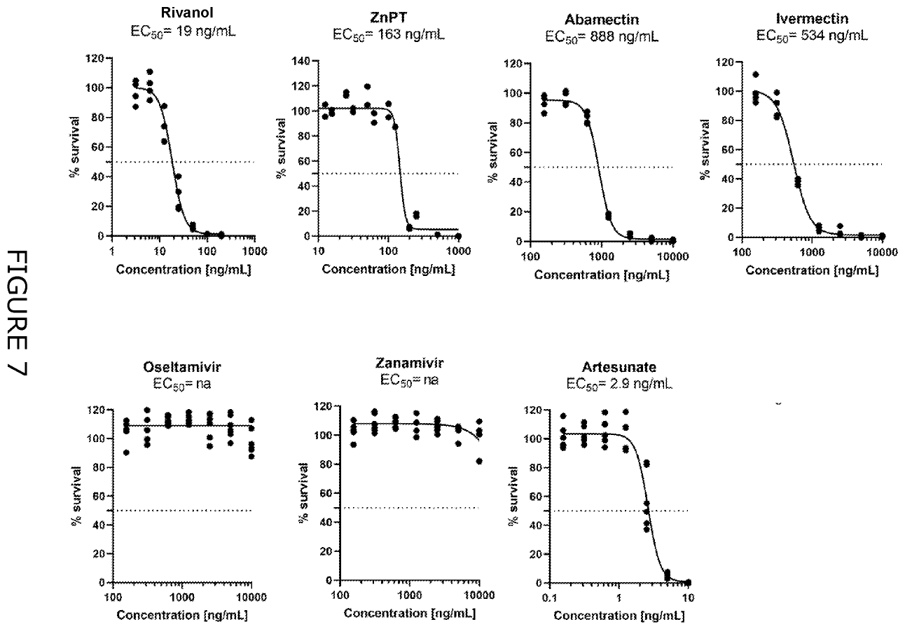

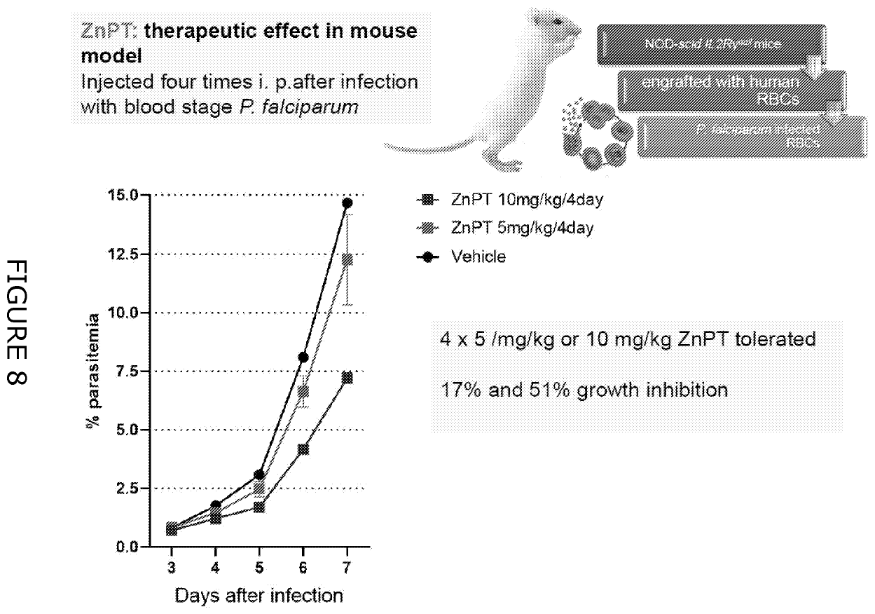

The structural basis of PfCyRPA α2-6-Neu5Ac lectin activity by molecular modelling, which was subsequently validated by PfCyRPA mutagenesis studies is shown in FIG. 2. Two Neu5Ac binding sites were identified (FIGS. 3 and 4), site 1, was identified adjacent to the PfCyRPA invasion blocking monoclonal antibody (mAb) C12 epitope (Favuzza, Guffart et al. 2017, supra), as shown in the PfCyRPA—mAb C12 co-crystal structure (FIG. 2B). When α2-6-SLN-Ac was docked into a box placed at Neu5Ac binding site 1, PfCyRPA residues belonging to blades 2 and 3 (E148, 1149, S152, 1155 and G209) were predicted to be involved in the major binding interactions within the α2-6-SLN-Ac-PfCyRPA complex (FIGS. 2A and B; FIG. 4).

The progenitor Plasmodium reichenowi CyRPA (PrCyRPA) does not have the same preference for Neu5Ac-α-(2-6)-Gal-β-(1-4)-GlcNAc instead binding both α-(2-3) and α-(2-6) sialic acid and both Neu5Ac and Neu5Gc (FIGS. 1A and B). Two amino acids within the region identified in FIG. 2 were found to be different between PfCyRPA and PrCyRPA (H154 and G209). Mutations to swap these residues between PfCyRPA and PrCyRPA demonstrate that the two point mutations in the PfCyRPA progenitor may have been sufficient for the adaptation of PfCyRPA towards higher affinity binding of human erythrocytes (FIG. 1B).

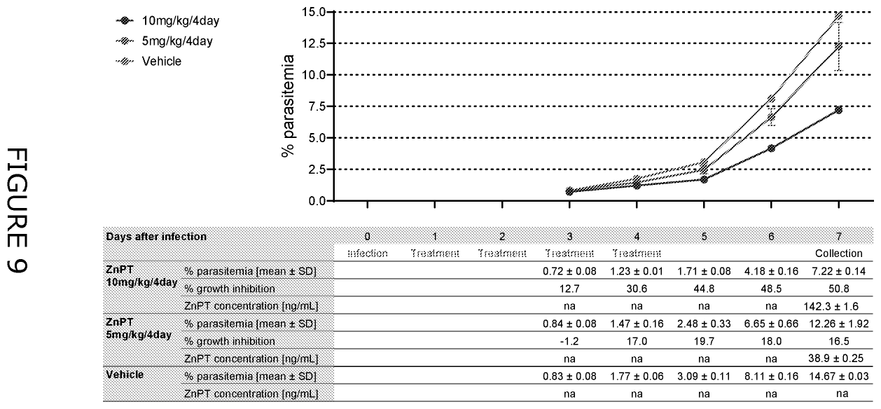

Fragments of PfCyRPA were used to further confirm the location of the lectin sites on PfCyPRA (FIG. 5). Binding of α2-6-SLN-Ac, α2-6-SLN-Gc and α2-6-Biant-Ac to PfCyRPA fragments were tested confirming the lectin site was in Fragment 2 (FIG. 5). The positions of amino acid residues implicated in lectin activity of fragment 2 are marked (FIG. 5).

Erythrocyte membrane ghosts were immobilized onto three flow cells of a BIAcore sensor chip. Flow cell 2 was treated with buffer only, flow cell 3 was treated with Sialidase that removes Neu5Ac with all linkages (α2-3,6,8,9; NEB) and Flow cell 4 was treated with sialidase that specifically removes α2-6 linked Neu5Ac. Removal of α2-6Neu5Ac or all sialic acid from erythrocyte membrane proteins reduced the affinity of PfCyRPA (146.1 nM to untreated) for the immobilized erythrocyte ghost membranes by 104-fold (15.16 μM) and 438-fold (63.97 μM), respectively (Table 1).

TABLE 1
AFFINITY CHANGE IN PFCYRPA BINDING TO SIALIDASE
TREATED ERYTHROCYTE (RBC) MEMBRANE GHOSTS.

| | RBC Ghosts + | RBC Ghosts + | RBC Ghosts + |
|---|---|---|---|
| | sialidase buffer | 2-6Sialidase | 2-3, 6, 8, 9Sialidase |
| PfCyRPA | 146.1 nM ± 12.8 | 15.16 μM ± 0.99 | 63.97 μM ± 11.4 |
| (KD) | | | |

### Methods

### Expression and Purification of PfCyRPA

Recombinant secreted P. falciparum wild type CyRPA was expressed essentially as described (Dreyer, Matile et al. 2012, supra; (Favuzza, Guffart et al. 2017, supra) as histidine-tagged fusion proteins comprising the CyRPA sequences without the signal sequence (aa 1-28). In brief, the expression vector pcDNA3.1_BVM_CyRPA(26-362)_His6 (Favuzza, Guffart et al. 2017, supra) was used to express the codon-optimized P. falciparum PF3D7_0423800 sequences as fusion protein with the bee-venom melitin signal sequence to allow secretion of the C-terminally His6tagged PfCyRPA into the cultivation medium. Expression vectors coding for PfCyRPA single amino acid variants were generated by site-directed mutagenesis (GenScript Biotech, Leiden, Netherlands). Plasmids were amplified in E. coli strain Top10 (Life Technologies) grown in LB medium under 100 mg/mL ampicillin selection and used for transfection of FreeStyle 293 F cells (Invitrogen, R790-07), a variant of human embryonic kidney HEK cells. Cells were cultured in suspension in serum-free medium (FreeStyle 293 Expression Medium, Thermo Fisher Scientific, Waltham, MA). 72 hr. post-transfection, cells were removed by filtration and the supernatant was concentrated and the His6-tagged recombinant fusion proteins were purified by immobilized metal ion affinity chromatography on a HisTrap HP column (GE-Healthcare).

### Glycan Array Analysis of Purified PfCyRPA.

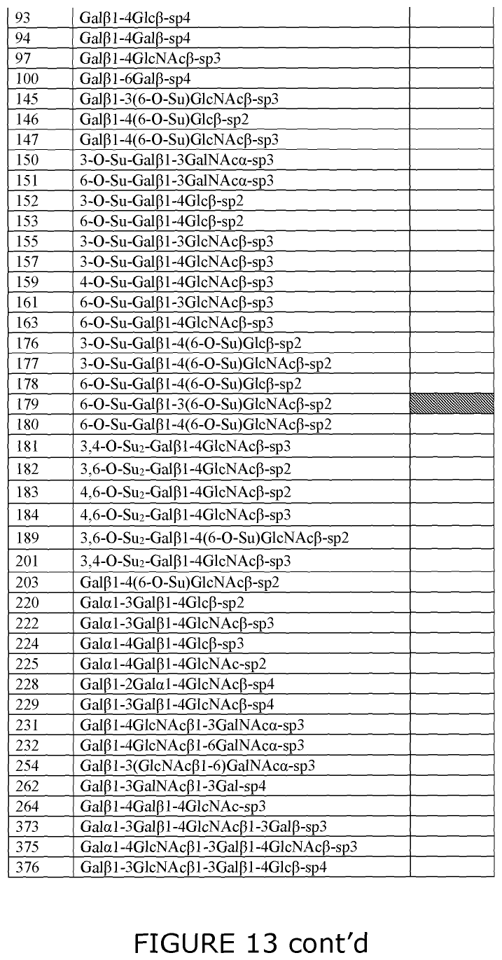

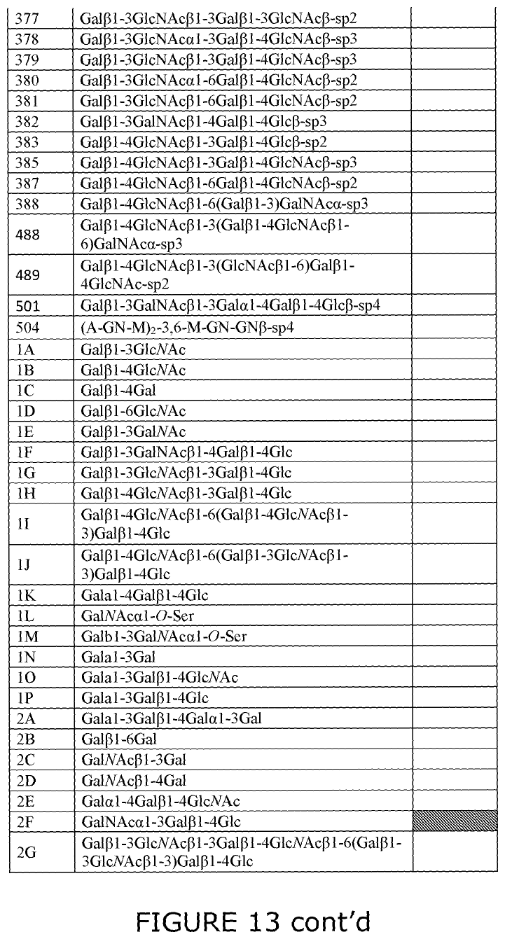

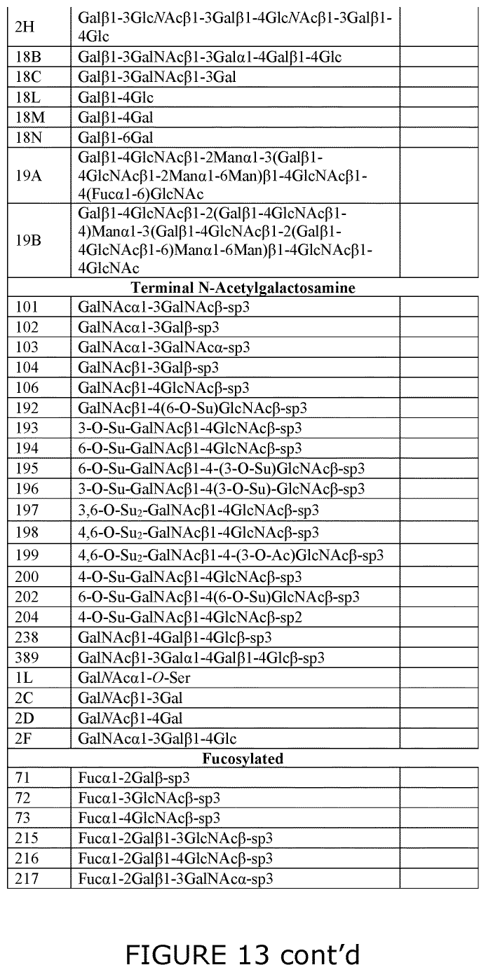

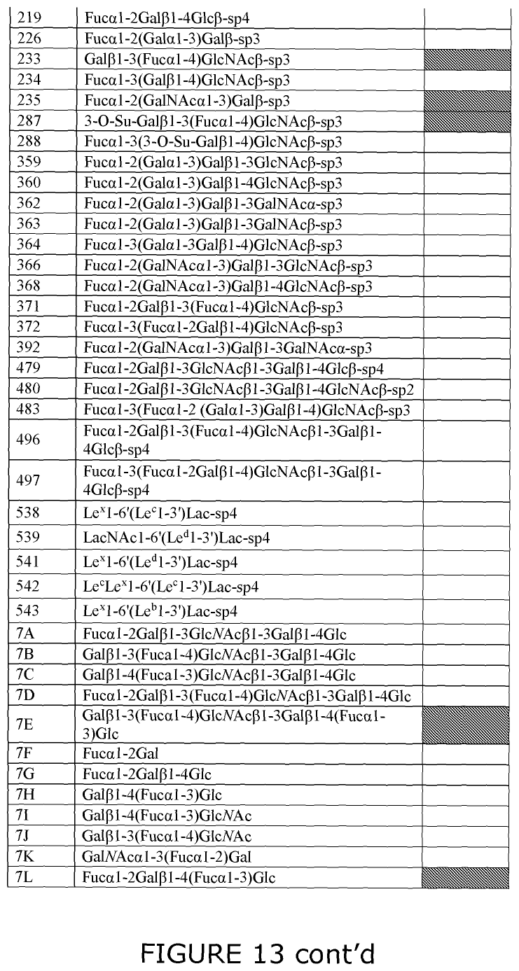

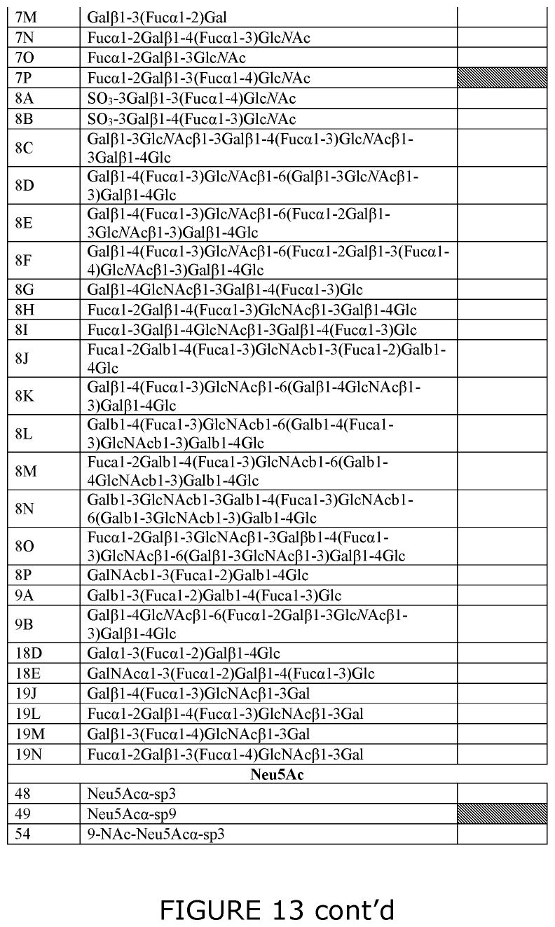

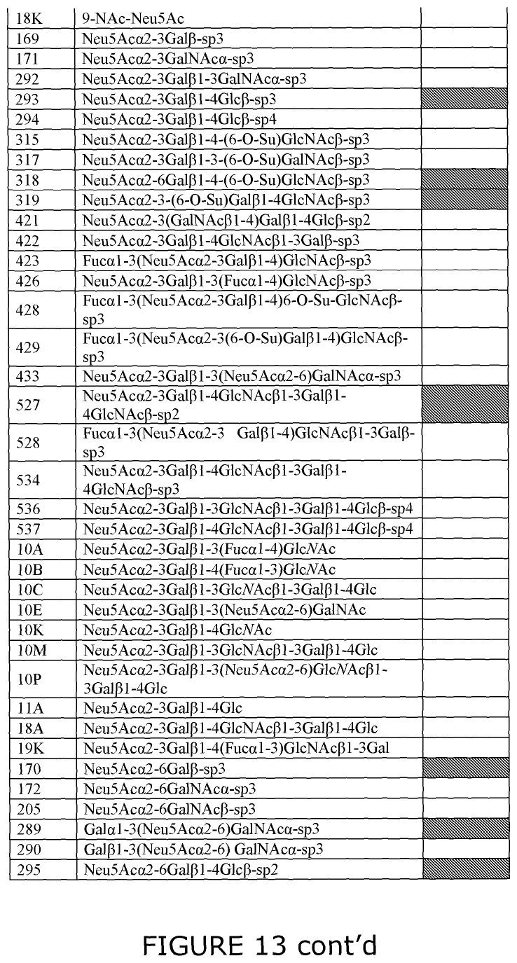

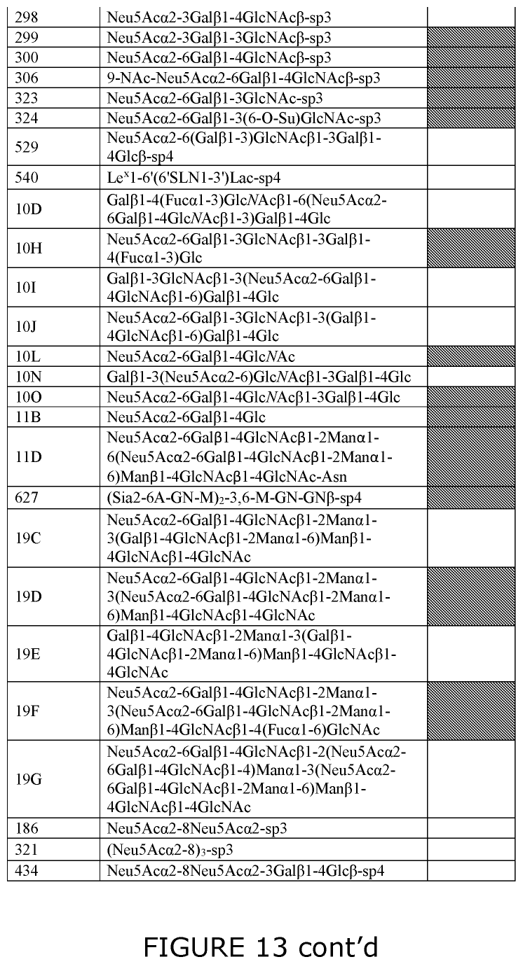

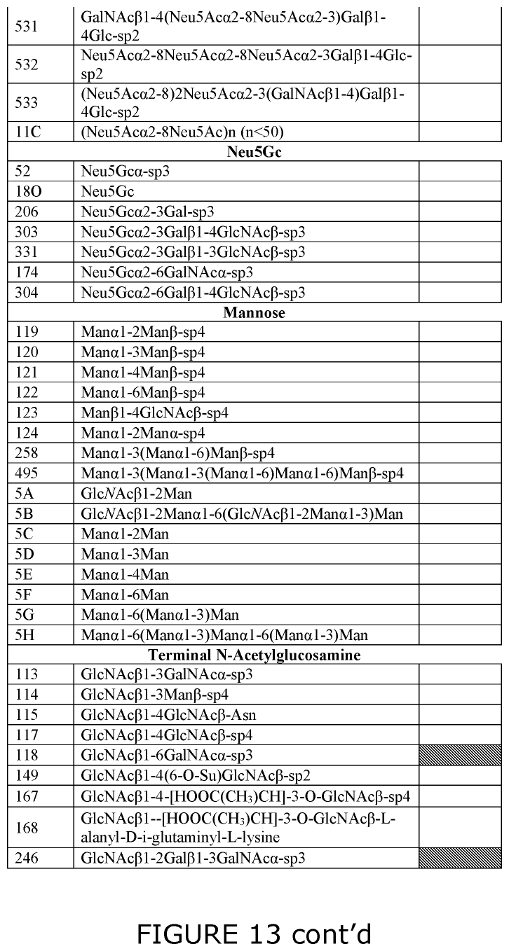

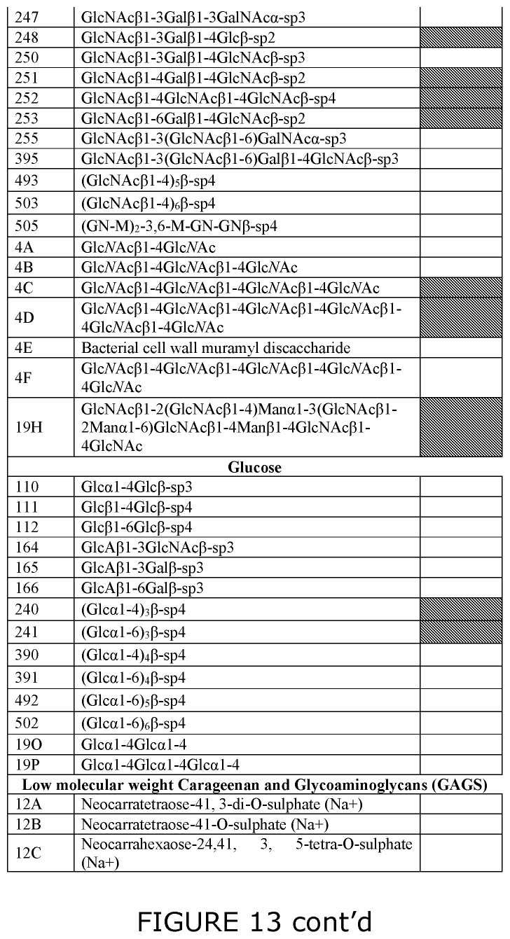

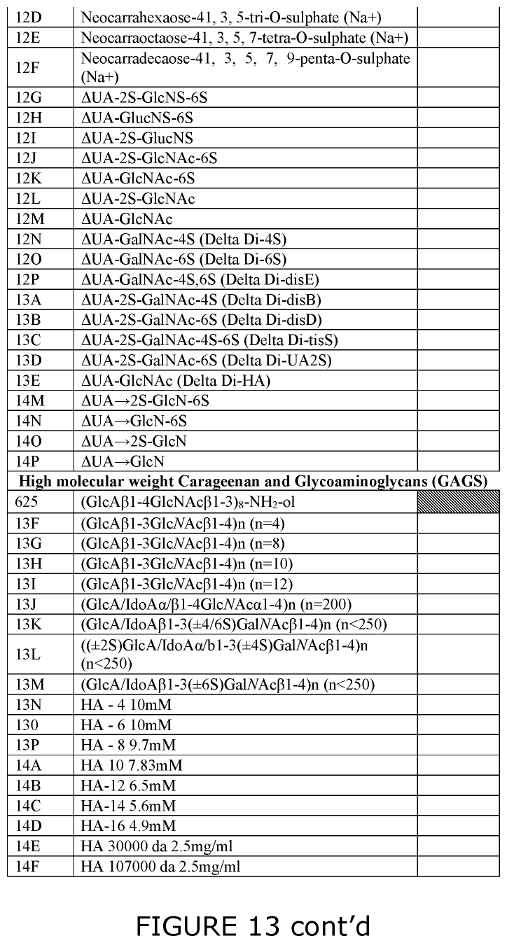

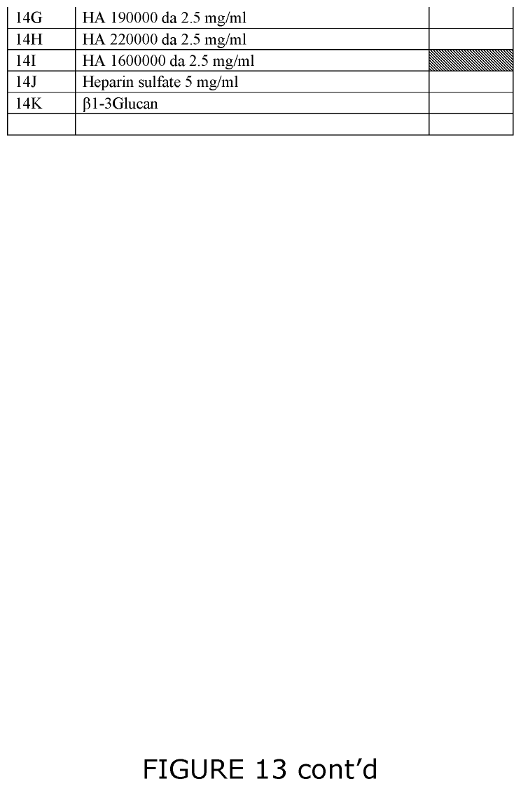

Glycan array slides were printed as previously described (Waespy, Gbem et al. 2015. PLoS Negl Trop Dis 9(10): e0004120). The Glycan array binding experiments were performed and analyzed as previously described (Shewell, Harvey et al. 2014. Proc Natl Acad Sci USA 111(49): E5312-5320). Briefly 2 μg of PfCyRPA in 1×PBS containing 1 mM MgCl₂ and 1 mM CaCl₂ was pre-complexed with mouse anti-His antibody (Cell signaling), Alexa555 rabbit anti-mouse IgG and Alexa555 goat anti-rabbit IgG (Thermo-Fisher) and allowed to bind to a pre-blocked (1% bovine serum albumin in PBS) for 15 minutes. Slides were washed three times for 2 minutes in 1×PBS, dried by centrifugation and scanned with Innopsys Innoscan 1100AL and analyzed using MAPIX software package and Microsoft Excel for statistical analysis (Student's unpaired t-test of fluorescence of background spots vs fluorescence of glycan printed spots). Results are from three arrays and total of 12 data points per glycan. See FIG. 13 for a full list of glycans.

### SPR Analysis of Purified PfCyRPA.

Purified PfCyRPA and PfCyRPA point mutants and PrCyRPA at 50 μg/mL were immobilized onto a series S CM5 chip using a Biacore S200 system (GE) at a flow rate of 5 μL/minute for 10 minutes in Sodium acetate pH4.0 using a modification of a method previously described (DuMont, Yoong et al. 2013. Proc Natl Acad Sci USA 110(26): 10794-10799). Initial glycans screens were done at a range of 160 nM to 100 μM, with the range refined for each interaction with a minimum concentration range of 1.6 nM-1 μM (2-6SLN compounds) performed. Antibody screening was performed with a concentration range of 1.6 nM-1 μM.

### Surface Plasmon Resonance (SPR) Analysis of PfCyRPA Binding to Red Blood Cell Ghosts.

Red blood cell membrane ghosts were prepared and solubilized as previously described (Shewell, Day et al., 2020. Sci Adv 6(21): eaaz4926). CHAPS solubilized membranes were immobilized onto flow cells 2-4 of a CM5 sensor chip (Cytiva) using a Biacore T200 (Cytiva) at pH 6.0 sodium acetate for 10 minutes at a flow rate of 5 μL/min. Erythrocyte membranes were tested with MAA (20 μg/mL; EY Laboratories), SNA (10 μg/mL; EY Laboratories) and CD147 antibody (TRA-1-85, 20 μg/mL; DSHB) before and after treatment with sialidase or buffer alone. Flow 2 was treated with the α2-6 sialidase buffer (50 mM sodium phosphate with 1 mM CMP) at a flow rate of 5 μL/min for 50 minutes at 37° C. Flow 3 was treated with the α2-3,6,8,9 sialidase (NEB) in NEB glycosidase buffer 1 at a flow rate of 5 μL/min for 50 min at 37° C. Flow 4 was treated with the α2-6 sialidase (1 mg/mL) in buffer (50 mM sodium phosphate with 1 mM CMP) at a flow rate of 5 μL/min for 50 min at 37° C. PfCyRPA was analyzed on the treated membranes at concentrations ranging from 1.6 nM to 50 μM.

### Expression of PfCyRPA Fragments on the Surface of HEK Cells.

293 HEK cells (ATCC, CRL-1573) expressing PfCyRPA fragments on the cell surface were generated essentially as described previously (Dreyer, Matile et al., 2012. J Immunol 188, 6225-6237). Briefly, PFD1130w-derived DNA sequences coding for fragments of PfCyRPA were amplified by PCR from the BVM_PFD1130W_FLAG_GP_His plasmid 1. These expression vectors allow the anchoring of the protein of interest on the cell surface via the transmembrane domain of mouse glycophorin-A (GP). In addition, they contain the secretion signal of bee-venom melittin, a FLAG tag located extracellularly, and a His6 tag located in the cytosol. The HEK cells were transfected using JetPEI transfection reagent (PolyPlus) according to the manufacturer's instructions. Transfectants were harvested 48 hr post-transfection and fixed for SPR analysis.

### SPR Analysis of Cells Expressing CyRPA Fragments

Whole 293 HEK cells expressing PfCyRPA fragments on the cell surface were fixed using 4% formaldehyde and washed three times with PBS and resuspended at 10⁷ cells/mL. Cells were immobilized onto a Series S C1 sensor chip using the C1 wizard methodology of the BIAcore S200 control system as described previously¹⁵. Cells were flowed at 5 μL per min for 900 sec to load the chip to saturation. 293 HEK cells not expressing PfCyRPA were loaded using the same methodology onto flow cell 1 to allow for double reference subtraction. Glycans were tested at a final concentration range of 160 nM-100 μM.

### Docking of N-Acetylneuraminic Acid (Sia, Neu5Ac), and α2-6- and α2-3-Sialyllactosamine (α2-6-SLN and α2-3-SLN) to PfCyRPA

To evaluate the potential N-acetylneuraminic acid (Neu5Ac) binding site, a docking experiment of PfCyRPA was performed using the AutoDock Vina protocol (Trott and Olson, 2010. J Comput Chem 31, 455-461). The X-ray crystal structure of PfCyRPA PDB 5EZN (10.2210/pdb5EZN/pdb) (Favuzza, Guffart et al. 2017, supra) was used for docking. It has been shown that AutoDock Vina has the highest scoring power among commercial and academic molecular docking programs (Wang, Sun et al., 2016. Phys Chem Phys 18, 12964-12975) implemented in the YASARA structure molecular modelling package (Ver. 16.46) (Krieger, Koraimann, et al., 2002. Proteins 47, 393-402). A blind docking experiment of Neu5Ac-αOH was set up using the entire PfCyRPA protein as a potential binding site (grid size 72.18×88.61×67.11 Å). Neu5Ac-αOH was generated using GLYCAM carbohydrate builder [Woods Group. (2005-2020) GLYCAM Web. Complex Carbohydrate Research Center, University of Georgia, Athens, GA (http://glycam.org)]. This initial blind docking procedure for Neu5Ac-αOH resulted in bound conformations near the predicted Asp-box. A local docking experiment of Neu5Ac-αOH was set up with a box size of 30×30×30 Å centered around the oxygen carbonyl atom at Thr-207 with 25 docking runs. Results from this local docking experiment revealed two clusters with bound Neu5Ac-αOH (see, FIG. 3) identified as the main and secondary Neu5Ac binding sites. Two more complex Neu5Ac-containing glycans (α2-6-SLN-Ac and α2-3-SLN-Ac) were docked into the identified main Neu5Ac binding site using the same procedures as described above.

### Example 2

### 2,6 Sialic Acid on Human RBCs is Required for Malaria Replication in Plasmodium Falciparum Blood Stage Culture in Human Red Blood Cells.

The key role of PfCyRPA binding to α2-6Neu5Ac in P. falciparum replication was supported by treatment of red blood cells with free glycans (FIGS. 6 A and B), an α2-6 specific sialidase (FIG. 6C) or with a PfCyRPA recognizing monoclonal antibody that can directly compete with glycan binding (Mab C12) (FIG. 6D-E). Parasitized erythrocytes (trophocoyte stage) treated with α2-6 Neu5Ac/Gc biantennary glycan structures, α2-6 sialidase or blocked with a PfCyRPA Mab that targets near the glycan binding site showed a reduction in parasite replication in cultured human RBCs, as shown in FIG. 6.

MAb C12 was tested in vivo using a humanized mouse model system and was found to inhibit P. falciparum replication (FIG. 6F).

### Methods

P. falciparum Blood-Stage Culture

Parasites were cultured essentially as described previously (Matile and Pink 1990. Immunological methods. I. Lefkovits and B. Pernis. San Diego, Academic Press. 4: 221-234). The culture medium was supplemented with 0.5% AlbuMAX (Life Technologies) as a substitute for human serum (Dorn, Stoffel et al. 1995. Nature 374(6519): 269-271). Cultures were synchronized by sorbitol treatment (Lambros and Vanderberg 1979. J Parasitol 65(3): 418-420). Erythrocytes for passages were obtained from the Swiss Red Cross (Switzerland).

P. falciparum Blood-Stage Culture Inhibition with Free Biantennary Glycan

A dose response of Neu5Ac/Gc biantennary glycan was added to the malaria replication assay described above with concentrations ranging from 10 μg/mL to 10 mg/mL.

### Expression and Purification of α2-6 Sialidase

The DNA sequence encoding residues 18-514 of the multifunctional α(2,6) sialidase from Photobacterium sp. strain JT-ISH-224 (accession number BAF92026) (18Pd224ST) (Kang, Lim et al. 2015. PLoS One 10(7): e0133739) was synthesized with flanking NdeI/BamHI sites and with codon optimization for E. coli expression by Integrated DNA Technologies (IDT—Iowa, USA) in the vector pUCIDT-Kan. pUCIDT-Kan containing the 18Pd224ST gene was used as template in a PCR reaction using KOD Polymerase as per the manufacturer's instructions (Merck Millipore) to amplify the gene for cloning. The resulting PCR product was purified and digested with NdeI and BamHI (NEB) and ligated into the NdeI/BamHI digested expression vector pET-15b and transformed into E. coli DH5α electrocompetent cells. The 18Pd224ST pET-15b expression construct was confirmed by DNA sequencing before being transformed into electrocompetent BL21 (DE3) cells for protein expression. Expression and purification was performed as described by (Huynh, Li et al. 2014. Febs Letters 588(24): 4720-4729) with the following modifications: protein expression was induced at OD600 nm=0.4-0.6, cell pellets were resuspended in 20 mM Tris-HCl/0.5M NaCl pH 7.5), His-tag protein was purified using TALON Metal Affinity Resin (Takara) as per the manufacturer's instructions and purified enzyme was stored at −20° C.

### α2-6 Sialidase Treatment of Human RBCs

Human group O RBCs were washed three times in SAGM (9 g glucose monohydrate, 8.77 g NaCl, 0.169 g Adenine, 5.25 g Mannitol/L) to remove lysed cells. 1 mL of 0.5% RBCs (v/v) (~2.5×10⁷ cells) diluted in SAGM were treated with either 1 mg of purified 18P244ST, 1 mg of purified 18P244ST+500 μM cytidine 5′-monophosphate (CMP) (Sigma Aldrich) (final concentration) or 500 μM (CMP) (final concentration) for 90 minutes at 37° C. with gentle agitation. Treated RBCs were harvested at 1500×g for 5 minutes at room temperature, washed once with 1 mL PBS and resuspended to 1% in PBS. To confirm that treatment with the 18P244ST α2-6 sialidase was successful, hemagglutination assays were performed using 0.5 μg/mL SNA-1 with 100 μL of 0.5% RBCs in round bottom 96-well plates. Hemagglutination was observed after 2 hours at room temperature. Loss of agglutination with SNA-1 was observed for RBCs treated with 1 mg of purified 18P244ST+500 μM CMP indicating removal of α2-6 sialic acids.

### Parasite Growth Inhibition Caused by α2-6 Sialidase Treatment of Erythrocytes

Human erythrocytes infected with synchronized trophozoites of the P. falciparum 3D7 clone were washed three times and resuspended at a density of 0.5% (v/v) in PBS. They were treated at 37° C. for 90 minutes with 1 mg/mL of α2-6 sialidase and 0.5 mM CMP or with sialidase or CMP alone with gentle agitation. Parasitized erythrocytes were subsequently cultivated for 48 hours and final parasitemia was quantified by flow cytometry after staining of viable parasites with hydroethidine. Growth inhibition was calculated relative to a control with untreated erythrocytes.

P. falciparum Blood-Stage Culture Inhibition with MAb C12

A dose response of MAb C12 was added to the malaria replication assay described above with concentrations ranging from 100 μg/mL to 10 mg/mL.

P. falciparum Humanized Mouse Model

The in vivo parasite inhibitory activity of compounds was tested in the P. falciparum mouse infection model essentially as described (Jimenez-Diaz, Mulet et al. 2009. Cytometry A 75(3): 225-235; Dreyer, Matile et al. 2012, supra). Six- to eight-week old female NSG mice (The Jackson Laboratory) were injected almost daily with 0.75 mL of human erythrocytes suspended in RPMI 1640 solution and decomplemented human serum at 50% hematocrit. Twelve days after the start of blood injections, groups of humanized mice were infected intravenously with 3×10⁷ parasitized erythrocytes obtained from in vitro cultures of P. falciparum strain Pf3D7^{0087/N9} developed for growth in hE engrafted mice (Jimenez-Diaz, Mulet et al. 2009, supra). Subsequently mice received repurposed drugs by intraperitoneal injection. The degrees of engraftment and parasitemia in peripheral blood were measured by flow cytometry using SYTO-16 green fluorescent nucleic acid stain (Invitrogen) and an allophycocyanin-labeled rat anti-mouse erythrocyte TER-119 monoclonal antibody (BD Biosciences).

### Ethical Statement

All procedures involving living animals were performed in strict accordance with the Rules and Regulations for the Protection of Animal Rights (Tierschutzverordnung) of the Swiss Federal Food Safety and Veterinary Office. The protocol was granted ethical approval by the Veterinary Office of the county of Basel-Stadt, Switzerland (Permit Numbers: 2375 and 2303).

### Example 3

High Throughput SPR Screening Identifies Drugs that Bind to P. falciparum CyRPA.

Recombinant P. falciparum CyRPA protein (PfCyRPA) was tested for binding to 3141 compounds using surface plasmon resonance. Initial screening of the compounds at 1 μM identified 6 compounds that bound with high affinity (see, Table 2).

TABLE 2
COMPOUNDS FROM DRUG SCREEN WITH SUB
1 μM DISASSOCIATION CONSTANT (K_{D})
VALUES IDENTIFIED IN SPR STUDIES.

| Compound | K_{D} (nM) |
|---|---|
| Zinc Pyrithione | 0.833 ± 0.192 |
| 6,9-Diamino-2-ethoxyacridine-DL-lactate monohydrate | 2.46 ± 0.410 |
| (RIVANOL) | |
| Abamectin | 19.1 ± 1.17 |
| Neohesperidin dihydrochalcone | 24.6 ± 1.45 |
| Simvastatin | 31.4 ± 2.24 |
| Buspirone hydrochloride | 33.6 ± 4.31 |

To investigate the specificity of PfCyRPA binding to zinc pyrithione, zinc only, pyrithione without zinc and a distinct salt of pyrithione, sodium pyrithione, were tested in SPR studies to determine the affinity of binding to PfCyRPA (see, Table 3).

TABLE 3
COMPARISON OF THE AFFINITY OF 1-HYDROXY-2(1H)-
PYRIDINETHIONE, ZINC, ZINC PYRITHIONE AND SODIUM
PYRITHIONE INTERACTIONS WITH PFCYRPA.

| Compound | K_{D} |
|---|---|
| Zinc Pyrithione | 0.833 nM ± 0.192 |
| Sodium Pyrithione | 122.3 μM ± 31.3 |
| 1-Hydroxy-2(1H)-pyridinethione | 169.4 μM ± 24.3 |
| Zinc | 546.3 μM ± 147.1 |

Only zinc pyrithione had a high affinity interaction with PfCyRPA (see, Table 2).

Abamectin is a macrocyclic lactone based on the lactones produced by Streptomyces avermitilis. Abamectin is toxic to humans, however, there is a large library of compounds based on the Streptomyces macrocyclic lactones that have been synthesized. The present inventors screened 7 additional macrocyclic lactone structures related to abamectin to determine whether these related compounds, that are safe for human use, could also target PfCyRPA (shown in Table 4).

TABLE 4
COMPARISON OF THE AFFINITY OF MACROCYCLIC LACTONES
RELATED TO ABAMECTIN BINDING TO PFCYRPA.

| Compound | K_{D} (nM) |
|---|---|
| Ivermectin | 93.2 ± 14 |
| Epimametcin | 3042 ± 506 |
| Selameticin | 1203 ± 202 |
| Milbemycin | 967 ± 57 |
| Moxidectin | 13412 ± 521 |
| Doramectin | 7044 ± 2032 |
| Emamectin | NB |

NB = no binding at 100 μM

Ivermectin is a macrocyclic lactone drug that has been used in humans. Of the macrocyclic lactone drugs related to abamectin that were tested, Ivermectin was found to have a high affinity interaction with PfCyRPA (Table 4).

### Materials and Methods

### Purified PfCyRPA Protein

Purified PfCyRPA protein was produced as previously described (Dreyer, Matile et al. 2012, supra; Favuzza, Blaser et al. 2016, supra) as histidine-tagged fusion protein comprising the CyRPA sequence without the signal sequence (aa 1-28).

### Drug Libraries

A combination of two libraries, Microsource-CPOZ (2400 drugs) and ML Drug (741 drugs) libraries, including FDA approved drugs, were purchased from Compounds Australia (Compounds Australia; Griffith Institute for Drug Discovery, Griffith University, Building N75, Brisbane Innovation Park, Don Young Road, Nathan QLD 4111).

### Drugs for Post-Screen Assays

All drugs for secondary SPR screening were obtained from SIGMA Aldrich (St Louis, Missouri): Zinc pyrithione (catalog number PHR1401), Zinc sulfate (catalog number, Z0251), Sodium Pyrithione (catalog number H3261), 1-Hydroxy-2(1H)-pyridinethione (catalog number 902713), Abamectin (catalog number 31732), RIVANOL (catalog number D16606), Buspirone hydrochloride (catalog number PHR1917), Neohesperidin dihydrochalcone (catalog number N0399000), Simvastatin (catalog number S0650000); with the exception of the additional Abamectin-related macrocyclic lactone drugs shown in Table 4, which were obtained from Merck.

### Repurposed Drug Screen Against Recombinant PfCyRPA

SPR analyses were performed using a BIAcore S200 System (GE Healthcare Life Sciences, Parramatta, NSW AUS). Samples were analyzed at 25° C. in phosphate buffered saline (PBS), at a flow rate of 10 μL/min. A combination of two libraries, including FDA approved drugs, Microsource-CPOZ (2400 drugs) and ML Drug (741 drugs), were purchased from Compounds Australia. Each drug was made up to 1 μM in 10% DMSO in a 384 well plate just prior to use in the BIAcore S200 system. A new biosensor chip was made for each 384 well plate screened. A single concentration injection screen binding assay was performed. Positive binding was defined by a response unit shift equal to the molecular weight corrected response units of the positive control glycan Neu5Acα2-6Galβ1-4GlcNAc (2-6SLN) on the stability of binding phase of the dissociation cycle. “Hits” were rescreened across a concentration range of 1.6 nM to 1 μM to define the concentration ranges for identifying the K_{D}. All compounds in this screen were rescreened to finalize the K_{D} with a concentration range of 0.16 to 100 nM.

### SPR Screening of Related Compounds

Recombinant PfCyRPA was immobilized as described above. Analysis was performed as per the rescreening protocol with concentrations ranging between 0.16 nm and 1 mM depending on compound solubility.

### Example 4

Assessing Capacity of Drugs that Bind PfCyRPA to Block the Binding of PfCyRPA to 2,6 Linked Sialic Acid Structures.

Recombinant Plasmodium falciparum CyRPA protein (PfCyRPA) was tested for binding to 3141 compounds with SPR. Screening identified 6 compounds that bound with nanomolar affinity (see Table 2 and 3).

These 6 compounds were rescreened for binding affinity and ability to block the binding of Neu5Acα2-6Galβ1-4GlcNAc (2-6SLN) to PfCyRPA in direct competition SPR experiments. The DPR competition studies demonstrated that 4 of the 6 compounds bound with sufficiently high affinity that they were able to completely block interactions between PfCyRPA and 2-6SLN. The compounds shown in Table 5 can block interactions between PfCyRPA and 2-6SLN.

TABLE 5
COMPOUNDS FROM THE DRUG SCREEN THAT
BLOCK 2-6 SLN BINDING TO PFCYRPA
IN SPR COMPETITION EXPERIMENTS.

| | | Blocking of |
|---|---|---|
| Compound | K_{D} (nM) | 2-6SLN binding |
| Zinc Pyrithione | 0.833 ± 0.192 | 98.2% ± 3.4 |
| RIVANOL | 2.46 ± 0.410 | 87.6% ± 9.7 |
| Abamectin | 19.1 ± 1.17 | 91.4% ± 6.8 |
| Ivermectin | 93.2 ± 14.1 | 67.3% ± 4.9 |
| Buspirone hydrochloride | 33.6 ± 4.31 | 77.4% ± 8.2 |

Competition analysis was performed with the compound injected first followed by addition of the 2-6SLN glycan. 100% blocking indicates that no 2-6SLN was bound by PfCyRPA in the presence of pre-bound compound with both molecules injected at 1 μM (saturating concentration).

### Materials and Methods

### Purified PfCyRPA Protein

Purified PfCyRPA protein was produced as previously described (Dreyer, Matile et al. 2012, supra; Favuzza, Blaser et al. 2016, supra) as histidine-tagged fusion proteins comprising the CyRPA sequences without the signal sequence (aa 1-28).

### SPR Competition

All drugs for secondary SPR competition screening were obtained from SIGMA Aldrich (St Louis, Missouri): Zinc pyrithione (catalog number PHR1401), Abamectin (catalog number 31732), RIVANOL (catalog number D16606), Buspirone hydrochloride (catalog number PHR1917); with the exception Ivermectin, which was obtained from Merck.

SPR Competition of Identified Drug with the 2-6SLN-PfCyRPA Interaction.

To determine if drugs with high affinity interactions with the PfCyRPA could inhibit the interaction with Neu5Acα2-6Galβ1-4GlcNAc (2-6SLN), a competition assay between PfCyRPA and compounds was performed as described Mubaiwa et al. (2017, Sci Rep. 7(1):5693). 2-6SLN, drug or drug and 2-6SLN were flowed at 1 μM over the immobilized PfCyRPA and the response units of the interactions were recorded. All SPR sensorgrams and results plots were analyzed with BIAcore S200 Evaluation Software (GE Healthcare Life Sciences).

### Example 5

Zinc Pyrithione can Block P. falciparum Replication in a Blood Stage Culture Assay with Human Red Blood Cells.

Zinc pyrithione (ZnPT) was identified to bind PfCyRPA with high affinity and inhibits the interaction between 2-6SLN and PfCyRPA. An in vitro human red blood cell blood infection stage parasite growth inhibition assay was performed (see, FIG. 7 and Table 6).

TABLE 6
ZNPT EC₅₀ IN HUMAN RED BLOOD CELL
PARASITE GROWTH INHIBITION ASSAY.

| Compound | EC₅₀ in RBC infection |
|---|---|
| Zinc Pyrithione | 163 ng/mL (513 nM) |

ZnPT was found to completely inhibit P. falciparum infection of human RBCs at concentrations of 500 ng/mL (1.57 μM; see, FIG. 7) with an EC50 of 163 ng/mL (513 nM).

### Materials and Methods

### Zinc Pyrithione

Zinc pyrithione (catalog number PHR1401) was obtained from SIGMA Aldrich (St Louis, Missouri).

P. falciparum Blood-Stage Culture

Parasites were cultured essentially as described previously (Matile and Pink 1990. Immunological methods. I. Lefkovits and B. Pernis. San Diego, Academic Press. 4: 221-234). The culture medium was supplemented with 0.5% AlbuMAX (Life Technologies) as a substitute forhuman serum (Dorn, Stoffel et al. 1995, supra). Cultures were synchronized by sorbitol treatment (Lambros and Vanderberg 1979, supra). Erythrocytes for passages were obtained from the Swiss Red Cross (Switzerland).

### In Vitro Growth Inhibition Assay (GIA)

In vitro growth inhibition assays with the laboratory-adapted P. falciparum 3D7 clone were performed essentially as described (Persson, Lee et al. 2006. J Clin Microbiol 44(5): 1665-1673). Synchronized trophozoites were adjusted to 0.5% parasitemia and then incubated for 48 hours with various concentrations of anti-CyRPA mAbs at 1% hematocrit. Each culture was set up in triplicate in 96-well flat-bottomed culture plates. Final parasitemia was quantified by flow cytometry after staining of viable parasites with hydroethidine and inhibition calculated relative to infection control wells containing PBS only.

EC₅₀ values for repurposed drugs were determined in vitro by measuring incorporation of the nucleic acid precursor [³H]-hypoxanthine. Infected red blood cells were exposed to increasing concentrations of compounds in culture plates. After 48 hours of incubation, 0.5 μCi [³H]-hypoxanthine was added to each well. Cultures were incubated for further 24 hours before they were harvested onto glass-fiber filters and washed with distilled water. The radioactivity was counted using a Betaplate liquid scintillation counter. The results were recorded as counts per minute per well at each compound concentration and expressed as percentage of the untreated controls. For each compound a four-parameter sigmoidal dose-response curve was fitted to the relationship between log₁₀(compound concentration) and % inhibition, and then used to interpolate EC₅₀ values. Data were processed and analyzed using GraphPad Prism 7.

### Example 6

Ivermectin and Abamectin can Block P. falciparum Replication in a Blood Stage Culture Assay with Human Red Blood Cells

Abamectin was identified to bind PfCyRPA with high affinity and inhibits the interaction between 2-6SLN and PfCyRPA and the related compound Ivermectin was identified to have similar binding and clocking properties. An in vitro human red blood cell blood infection stage parasite growth inhibition assay was performed (see, FIG. 7 and Table 7).

TABLE 7
ABAMECTIN/IVERMECTIN EC₅₀ IN HUMAN RED BLOOD
CELL PARASITE GROWTH INHIBITION ASSAY.

| Compound | EC₅₀ in RBC infection | |
|---|---|---|
| Abamectin | 888 ng/mL | (1001 nM) |
| Ivermectin | 534 ng/mL | (610 nM) |

Abamectin was found to completely inhibit P. falciparum infection of human RBCs at concentrations of 4000 ng/mL (4.50 μM; see, FIG. 7) with an EC50 of 888 g/mL (1001 nM).

Ivermectin was found to completely inhibit P. falciparum infection of human RBCs at concentrations of 2000 ng/mL (2.29 μM; see, FIG. 7) with an EC50 of 534 g/mL (610 nM).

### Materials and Methods

### Abamectin

Abamectin (catalog number 31732) and was obtained from SIGMA Aldrich (St Louis, Missouri) and Ivermectin was obtained from Merck.

P. falciparum Blood-Stage Culture

Parasites were cultured essentially as described previously (Matile and Pink 1990). The culture medium was supplemented with 0.5% AlbuMAX (Life Technologies) as a substitute for human serum (Dorn, Stoffel et al. 1995, supra). Cultures were synchronized by sorbitol treatment (Lambros and Vanderberg 1979, supra). Erythrocytes for passages were obtained from the Swiss Red Cross (Switzerland).

### In Vitro Growth Inhibition Assay (GIA)

In vitro growth inhibition assays with the laboratory-adapted P. falciparum 3D7 clone were performed essentially as described (Persson, Lee et al. 2006, supra). Synchronized trophozoites were adjusted to 0.5% parasitemia and then incubated for 48 hours with various concentrations of anti-CyRPA mAbs at 1% hematocrit. Each culture was set up in triplicate in 96-well flat-bottomed culture plates. Final parasitemia was quantified by flow cytometry after staining of viable parasites with hydroethidine and inhibition calculated relative to infection control wells containing PBS only.

EC₅₀ values for repurposed drugs were determined in vitro by measuring incorporation of the nucleic acid precursor [³H]-hypoxanthine. Infected red blood cells were exposed to increasing concentrations of compounds in culture plates. After 48 hours of incubation, 0.5 μCi [³H]-hypoxanthine was added to each well. Cultures were incubated for further 24 hours before they were harvested onto glass-fiber filters and washed with distilled water. The radioactivity was counted using a Betaplate liquid scintillation counter. The results were recorded as counts per minute per well at each compound concentration and expressed as percentage of the untreated controls. For each compound a four-parameter sigmoidal dose-response curve was fitted to the relationship between log₁₀(compound concentration) and % inhibition, and then used to interpolate EC₅₀ values. Data were processed and analyzed using GraphPad Prism 7.

### Example 7

Rivanol can Block P. falciparum Replication in a Blood Stage Culture Assay with Human Red Blood Cells.

RIVANOL was identified to bind PfCyRPA with high affinity and inhibits the interaction between 2-6SLN and PfCyRPA. An in vitro human red blood cell blood infection stage parasite growth inhibition assay was performed (see, FIG. 7 and Table 8).

TABLE 8
RIVANOL EC₅₀ IN HUMAN RED BLOOD CELL
PARASITE GROWTH INHIBITION ASSAY.

| Compound | EC50 in RBC infection |
|---|---|
| RIVANOL | 19 ng/mL (55.3 nM) |

RIVANOL was found to completely inhibit P. falciparum infection of human RBCs at concentrations of 100 ng/mL (291.1 nM; see, FIG. 7) with an EC₅₀ of 19 ng/mL (55.3 nM).

### Materials and Methods

### RIVANOL

RIVANOL (catalog number D16606) was obtained from SIGMA Aldrich (St Louis, Missouri).

P. falciparum Blood-Stage Culture

Parasites were cultured essentially as described previously (Matile and Pink 1990, supra). The culture medium was supplemented with 0.5% AlbuMAX (Life Technologies) as a substitute for human serum (Dorn, Stoffel et al. 1995, supra). Cultures were synchronized by sorbitol treatment (Lambros and Vanderberg 1979, supra). Erythrocytes for passages were obtained from the Swiss Red Cross (Switzerland).

### In Vitro Growth Inhibition Assay (GIA)

In vitro growth inhibition assays with the laboratory-adapted P. falciparum 3D7 clone were performed essentially as described (Persson, Lee et al. 2006, supra). Synchronized trophozoites were adjusted to 0.5% parasitemia and then incubated for 48 hours with various concentrations of anti-CyRPA mAbs at 1% hematocrit. Each culture was set up in triplicate in 96-well flat-bottomed culture plates. Final parasitemia was quantified by flow cytometry after staining of viable parasites with hydroethidine and inhibition calculated relative to infection control wells containing PBS only.

EC₅₀ values for repurposed drugs were determined in vitro by measuring incorporation of the nucleic acid precursor [³H]-hypoxanthine. Infected red blood cells were exposed to increasing concentrations of compounds in culture plates. After 48 hours of incubation, 0.5 μCi [³H]-hypoxanthine was added to each well. Cultures were incubated for further 24 hours before they were harvested onto glass-fiber filters and washed with distilled water. The radioactivity was counted using a Betaplate liquid scintillation counter. The results were recorded as counts per minute per well at each compound concentration and expressed as percentage of the untreated controls. For each compound a four-parameter sigmoidal dose-response curve was fitted to the relationship between log₁₀(compound concentration) and % inhibition, and then used to interpolate EC₅₀ values. Data were processed and analyzed using GraphPad Prism 7.

### Example 8

Zinc Pyrithione can Reduce P. falciparum Replication in a Humanized Mouse Model of Blood Stage Infection and a Method of Measurement of Zinc Pyrithione in Samples Using Dual Zinc Pyrithione Binding Proteins.

Zinc pyrithione (ZnPT) was identified to bind PfCyRPA with high affinity and inhibits the interaction between 2-6SLN and PfCyRPA and could block in vitro human red blood cell blood infection with an EC₅₀ of 163 ng/mL (513 nM). This compound was tested in vivo using a humanized mouse model system (see, FIGS. 8 and 9).

ZnPT was found to reduce parasitemia of the mice by 51% when injecting 10 mg/kg i.p. for the first 4 days after infection.

To determine the level of ZnPT in serum a methodology using two ZnPT binding proteins was developed. The two proteins used were PfCyRPA and New Delhi Metalo-β-lactamase 2 (NDM2, New Delhi metallo-beta-lactamase 2, Yaramah, Jen et al, 2020. Biochem Biophys Res Commun 524(3):555-560), which the inventors observed to also bind with high affinity to ZnPT (unpublished data). The relative affinities if PfCyRPA and NDM2 for zinc pyrithione and related compounds are shown in Table 9.

TABLE 9
PYRITHIONE AND ZINC INTERACTIONS WITH NDM2.

| Compound | CyRPA K_{D} | NDM2 K_{D} |
|---|---|---|
| Zinc Pyrithione | 0.833 nM ± 0.192 | 38.5 nM ± 6.4 |
| Sodium Pyrithione | 122.3 μM ± 31.3 | 789.2 μM ± 98.4 |
| 1-Hydroxy-2(1H)-pyridinethione | 169.4 μM ± 24.3 | 894.0 μM ± 143 |
| Zinc | 546.3 μM ± 147 | 317 μM ± 113 |

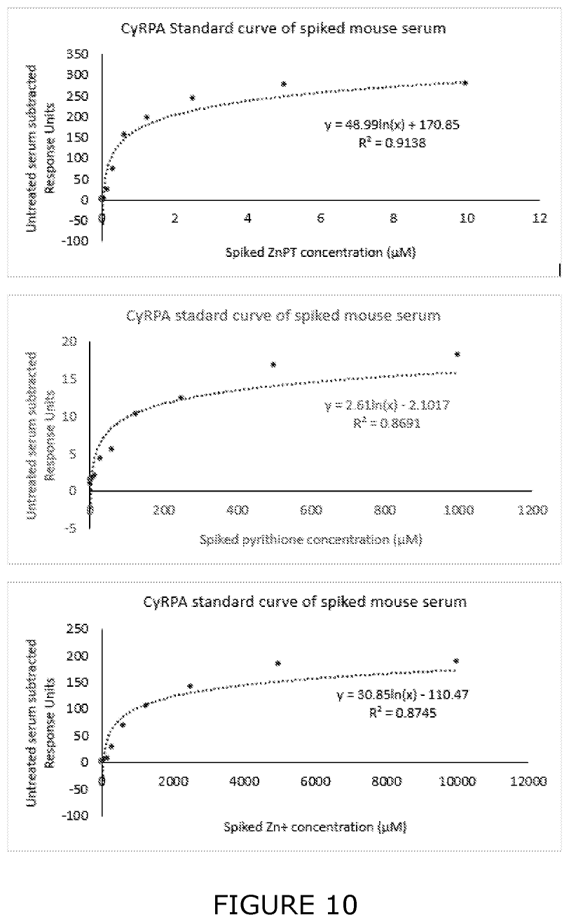

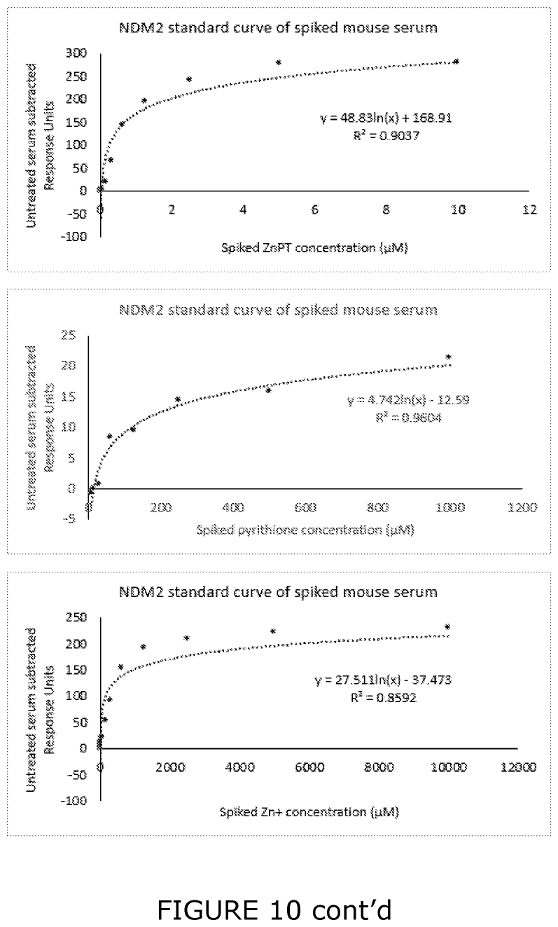

Based on these binding profiles standard curves for ZnPT, PT and zinc were established in mouse serum and used to estimate the concentration of ZnPT serum for each treated mouse tested and untreated control mice (see, Table 10 and FIG. 10).

Serum concentration at the point of testing (7 days post infection) was below the EC₅₀ for ZnPT of 163 ng/mL (51 3 nM).

TABLE 10
SERUM ZNPT CONCENTRATIONS IN MICE TREATED WITH
10 MG/KG ZNPT FOR 4 DAYS POST INFECTION.

| | Vehicle treated subtracted ZnPT serum level |
|---|---|
| Mouse 1 | 141.6 ng/mL (446 nM) |
| Mouse 2 | 143.8 ng/mL (453 nM) |

### Materials and Methods

P. falciparum Humanized Mouse Model

The in vivo parasite inhibitory activity of compounds was tested in the P. falciparum mouse infection model essentially as described (Jimenez-Diaz, Mulet et al. 2009. Cytometry A 75(3): 225-235; Dreyer, Matile et al. 2012, supra). Six to eight week old female NSG mice (The Jackson Laboratory) were injected almost daily with 0.75 mL of human erythrocytes suspended in RPMI 1640 solution and decomplemented human serum at 50% hematocrit. Twelve days after the start of blood injections, groups of humanized mice were infected intravenously with 3×10⁷ parasitized erythrocytes obtained from in vitro cultures of P. falciparum strain Pf3D7^{0087/N9} developed for growth in hE engrafted mice (Jimenez-Diaz, Mulet et al. 2009, supra). Subsequently mice received repurposed drugs by intraperitoneal injection. The degrees of engraftment and parasitemia in peripheral blood were measured by flow cytometry using SYTO-16 green fluorescent nucleic acid stain (Invitrogen) and an allophycocyanin-labeled rat anti-mouse erythrocyte TER-119 monoclonal antibody (BD Biosciences).

### Ethical Statement

All procedures involving living animals were performed in strict accordance with the Rules and Regulations for the Protection of Animal Rights (Tierschutzverordnung) of the Swiss Federal Food Safety and Veterinary Office. The protocol was granted ethical approval by the Veterinary Office of the county of Basel-Stadt, Switzerland (Permit Numbers: 2375 and 2303).

SPR Measurement of Serum ZnPT Concentrations Using a Standard Curve of ZnPT Spiked into Untreated Mouse Serum.

SPR with immobilized PfCyRPA was performed as described in Examples 3 and 4. NDM2 was immobilized using the same methodology as PfCyRPA.

Standard curves were run across the following concentrations:
ZnPT—0.61 nM to 10 μM
PT—61 nM to 1 mM
Zinc—0.61 μM to 10 mM

### Example 9

Ivermectin can Reduce P. falciparum Replication in a Humanized Mouse Model of Blood Stage Infection Proteins.

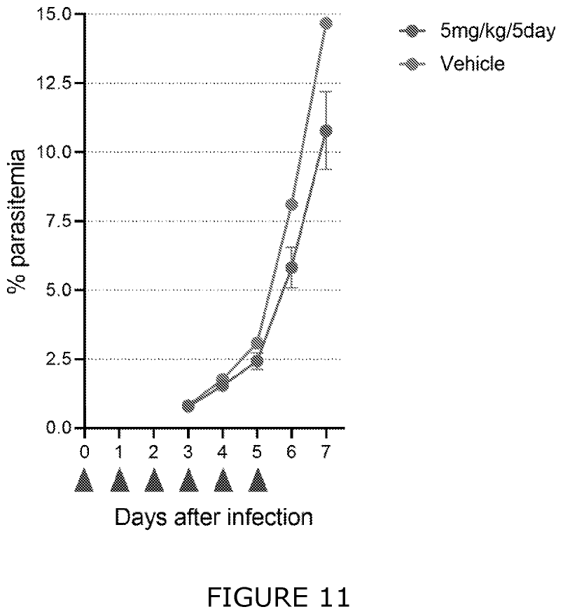

Ivermectin was identified to bind PfCyRPA with high affinity and inhibits the interaction between 2-6SLN and PfCyRPA and could block in vitro human red blood cell blood infection with an EC₅₀ of 19 ng/mL (55.3 nM). This compound was tested in vivo using a humanized mouse model system (see, FIG. 11).

Ivermectin was found to reduce parasitemia of the mice by 26% when injecting 5 mg/kg I.P. for the first 5 days after infection.

### Example 10

### Zinc Pyrithione Absorption Study in Humans.

In an ex vivo model of P. falciparum replication in human red blood cells and in a in vivo humanized mouse model, the present inventors demonstrated that zinc pyrithione delivered IP could inhibit parasite replication. Zinc pyrithione (ZnPT) is commonly used as a topical application to treat dandruff in formulations such as shampoos. It is common for such shampoos to contain 1% w/v zinc pyrithione as the active ingredient. Although there have been extensive studies in animals and humans, but information on the concentration of zinc pyrithione in human serum after topic application of zinc pyrithione is not readily available. To determine the concentration of zinc pyrithione in human serum after topical application of shampoos to contain 1% w/v zinc pyrithione, a study was conducted in humans and a methodology was developed for direct measurement in solution using two independent proteins that bind zinc pyrithione with high affinity and specificity. The serum concentration of ZnPT was measured at day 1, 3, 4, 5, 10, shown in Table 11. The highest concentration of ZnPT was observed on day 5 (the day after the three consecutive days of treatment had ceased). By day 5, 6 of the 10 individuals treated with the topical 1% ZnPT product achieved a serum concentration above the EC₅₀ observed for ZnPT in P. falciparum inhibition assays (Table 6).

TABLE 11
SERUM CONCENTRATIONS OF ZINC PYRITHIONE IN
TEN HUMAN SUBJECTS THROUGHOUT THE PROTOCOL.

| | Serum 1 | Serum 2 | Serum 3 | Serum 4 | Serum 5 |
|---|---|---|---|---|---|
| Day 1 | 0.03 μM ± | 0.02 μM ± | 0.08 μM ± | 0.03 μM ± | 0.01 μM ± |
| | 0.05 | 0.002 | 0.003 | 0.01 | 0.001 |
| Day 3 | 0.04 μM ± | 0.88 μM ± | 0.24 μM ± | 0.81 μM ± | 0.02 μM ± |
| | 0.07 | 0.2 | 0.05 | 0.2 | 0.1 |
| Day 4 | 0.66 μM ± | 1.8 μM ± | 0.84 μM ± | 1.0 μM ± | 0.10 μM ± |
| | 0.08 | 0.3 | 0.06 | 0.16 | 0.1 |
| Day 5 | 2.7 μM ± | 4.3 μM ± | 1.6 μM ± | 1.5 μM ± | 0.22 μM ± |
| | 0.04 | 0.5 | 0.22 | 0.31 | 0.1 |
| Day 10 | −0.07 μM ± | 0.33 μM ± | 1.0 μM ± | 1.2 μM ± | 0.02 μM ± |
| | 0.2 | 0.2 | 0.21 | 0.42 | 0.01 |
| | Serum 6 | Serum 7 | Serum 8 | Serum 9 | Serum 10 |
| Day 1 | 0.001 μM ± | 0.01 μM ± | 0.02 μM ± | 0.04 μM ± | 0.004 μM ± |
| | 0.00004 | 0.0003 | 0.001 | 0.003 | 0.0003 |
| Day 3 | 0.10 μM ± | 0.84 μM ± | 0.02 μM ± | 0.22 μM ± | 0.15 μM ± |
| | 0.02 | 0.05 | 0.04 | 0.003 | 0.03 |
| Day 4 | 0.21 μM ± | 0.96 μM ± | 0.09 μM ± | 0.30 μM ± | 1.1 μM ± |
| | 0.02 | 0.06 | 0.03 | 0.009 | 0.04 |
| Day 5 | 0.38 μM ± | 1.5 μM ± | 0.26 μM ± | 0.49 μM ± | 1.5 μM ± |
| | 0.08 | 0.08 | 0.1 | 0.03 | 0.07 |
| Day 10 | 0.007 μM ± | 1.2 μM ± | 0.11 μM ± | 0.14 μM ± | 0.98 μM ± |
| | 0.008 | 0.05 | 0.0002 | 0.004 | 0.11 |

### Materials and Methods

### Design of the Zinc Pyrithione Absorption Study in Humans

Informed written consent was obtained from participants above 18 years of age, who had not used any products containing zinc pyrithione in the last 12 months, who were not currently taking antibiotics or antiparasitic medication, who were not pregnant and who were not currently involved in a trial or within 30 days of finishing a trial (n=6). A blood and urine sample was collected from participants on day 1 before commencing use of a commercially available anti-dandruff shampoo (Head and Shoulders™, 1% ZPT) applied morning and evening for three consecutive days starting on day 2. Participants were instructed to completely wet hair, measure out 20 mL of anti-dandruff shampoo using a measuring cup provided, pour shampoo onto the scalp and massage shampoo into the scalp, leave shampoo on for 5-10 minutes using the timer provided then completely rinse off shampoo. Blood and urine samples were also collected on day 3 (two days of shampoo use), day 4 (three days of shampoo use), day 5 (day after shampoo use stops) and day 10 (five days after shampoo use has stopped). Serum was separated from blood collected in SST II Advance serum separation tubes (BD) according to the manufacturer's instructions. Sera and urine were batched and stored at −80° C. within 4 hours of collection.

### Ethical Statement

This human study was performed according to the National Statement on Ethical Conduct in Human Research (2007) with a protocol approved by the Griffith University Human Research Ethics Committee (Protocol number 2019/640).

### SPR Measurement of Serum ZnPT

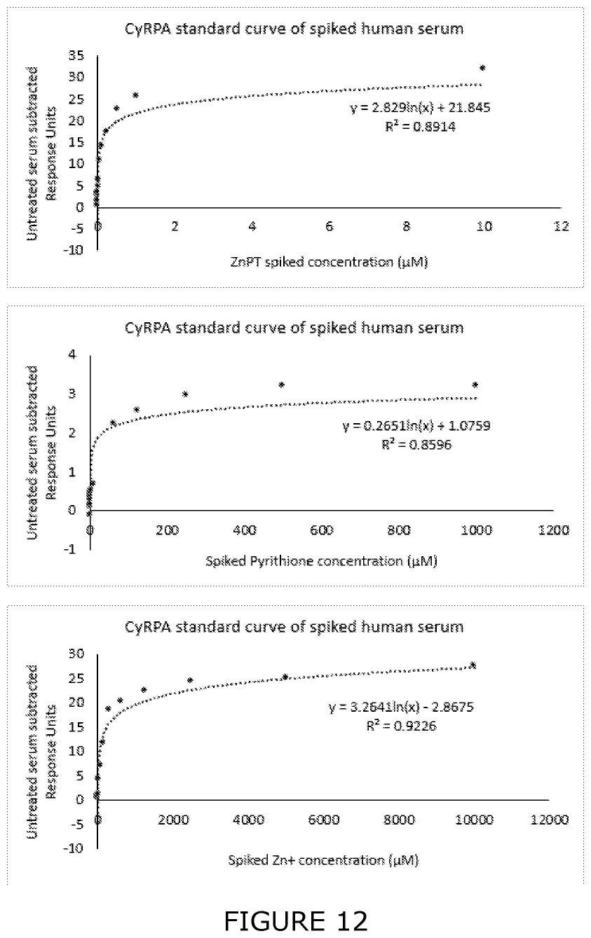

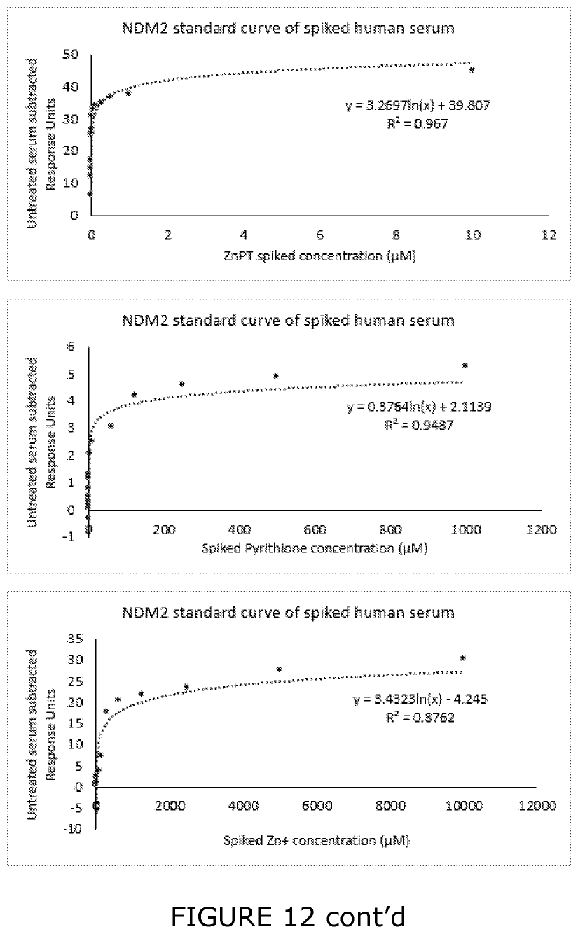

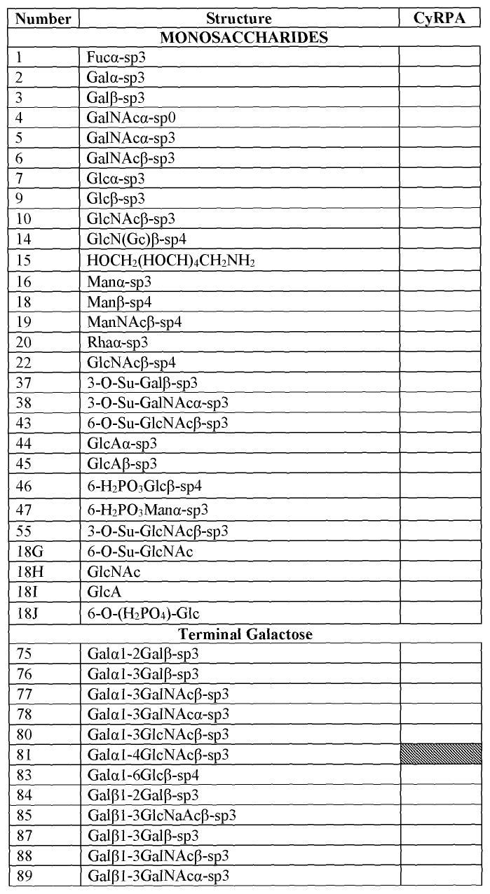

SPR measurement of serum ZnPT concentrations using a standard curve of ZnPT spiked into untreated normal human serum was performed as described in Example 8. The standard curves for human serum are shown in FIG. 12.

Standard curves were run across the following concentrations:
ZnPT—0.61 nM to 10 μM
PT—61 nM to 1 mM
Zinc—0.61 μM to 10 mM

### Example 11

Drugs that Bind to P. falciparum CyRPA Also Interact with CyRPA from P. vivax.

Orthologs of PfCyRPA have been found in the genomes of the human malaria parasite P. vivax and the primate pathogens P. knowlesi, P. cynomolgi, and P. reichenowi, but not in the sequenced genomes of other Plasmodium species (Favuzza, Guffart et al. 2017, supra). P. knowlesi, P. cynomolgi infections have also been found in humans (Müller M, Schlagenhauf P. Malar. J. 2014; 13:68). To investigate whether the drugs identified as active against PfCyRPA may also have utility in other human malaria species, recombinant P. vivax CyRPA protein (PvCyRPA) was tested for binding to the four inhibitory drugs identified for PfCyRPA: ZnPT, RIVANOL, Abamectin and Ivermectin by SPR analysis (see, Table 12).

TABLE 12
COMPOUNDS FROM PFCYRPA ANALYSIS TESTED
FOR DISASSOCIATION CONSTANT (K_{D}) VALUES
IDENTIFIED IN SPR STUDIES WITH PVCYRPA.

| Compound | K_{D} (nM) |
|---|---|
| Zinc Pyrithione | 50.83 ± 2.91 |
| 6,9-Diamino-2-ethoxyacridine-DL-lactate monohydrate | 6.98 ± 1.40 |
| (RIVANOL) | |
| Abamectin | 1143 ± 230 |
| Ivermectin | 160.7 ± 42.1 |

### Materials and Methods

### Purified PvCyRPA Protein

Purified PvCyRPA protein was produced as previously described (Dreyer, Matile et al. 2012, supra; Favuzza, Blaser et al. 2016, supra) as histidine-tagged fusion protein comprising the CyRPA sequence without the signal sequence (aa 1-23).

### Drugs for Post-Screen Assays

All drugs for secondary SPR screening were obtained from SIGMA Aldrich (St Louis, Missouri): Zinc pyrithione (catalog number PHR1401), Abamectin (catalog number 31732), RIVANOL (catalog number D16606) and Ivermectin was obtained from Merck.

### Repurposed Drug Screen Against Recombinant PvCyRPA

SPR analyses were performed using a BIAcore S200 System (GE Healthcare Life Sciences, Parramatta, NSW AUS). Samples were analyzed at 25° C. in phosphate buffered saline (PBS), at a flow rate of 10 μL/min. Drugs were tested across a concentration range of 1.6 nM to 1 μM to define the concentration ranges for identifying the K_{D}. SPR screening of related compounds.

### Example 12

Activity of Zinc Pyrithione Against a Panel of Plasmodium falciparum Strains.

Assessment of the in vitro effectiveness of zinc pyrithione in erythrocytic Plasmodium falciparum growth was performed with two panels of parasites: 1) A panel of drug resistant and sensitive P. falciparum field isolates adapted to laboratory culture. 2) A panel of induced drug resistant P. falciparum clones generated in the laboratory of Prof. David Fidock (Columbia University, USA). These two panels have defined levels of drug resistance outlined in Tables 13 and 14.

Additionally, the piperaquine- and chloroquine-resistant field isolate RF12 (named also PH1263-C in Ross et al. (2018, Nat Commun. 9(1):3314) obtained from the laboratory of Prof. David Fidock, Columbia University, USA) was used. IC₅₀ shifts were calculated from mean IC₅₀ values determined in n≥2 independent [³H]hypoxanthine incorporation experiments. Resistant (R) or sensitive (S) is indicated in some cases where the IC₅₀ fold shift have not been measured or published but the clinical resistance profile and/or the resistance genotype was reported previously (Ding et al., 2012. Malar J. 11:292). Specific culture conditions are required to measure IC₅₀s with SUL and were not measured in Chugh et al. (2015, Antimicrob Agents Chemother. 59(2): 1110-1118). The lighter gray highlight indicates that cross-resistance was expected from clinical studies but the fold IC₅₀ shift was <10 in vitro. The darker gray highlight indicates cases in which the fold IC₅₀ shift relative to NF54 is >10×. Abbreviations: chloroquine (CHQ), sulfadoxine (SUL), pyrimethamine (PYR), cycloguanil (CYC), mefloquine (MFQ), atovaquone (ATO), artesunate (ATS). ND, not defined.

Screening of the two panels of parasites with Zinc pyrithione found an IC50 range of between 273 nM and 620 nM as outlined in TABLES 15 and 16.

The data in Tables 15 and 16 show that zinc pyrithione inhibits P. falciparum growth in vitro in a range of drug resistant parasite regardless of the resistance mechanism including all currently known resistance forms. Zinc pyrithione also inhibited the growth of lab-derived P. falciparum resistant clones showing that zinc pyrithione likely acts through a mechanism of action that is not affected by resistance against validated antimalarial targets.

### Materials

ZNPT (batch #LRAC8770), Sigma PHR1401-5G, obtained on 25 Nov. 2023; Chloroquine diphosphate, Sigma (C-6628), Switzerland; Artesunate, Mepha Pharma AG, 4010 Basel (Switzerland).

### Methods

Parasite growth in the presence of antimalarial compounds was assessed in 2 independent experiments using the [3H]hypoxanthine incorporation assay and expressed by IC50 values [Snyder 2007]. The total incubation time per assay was 72 hours; radioactive hypoxanthine was added for the last 24 hours. The antimalarial drugs chloroquine and artesunate served as internal controls in every assay.

The disclosure of every patent, patent application, and publication cited herein is hereby incorporated by reference herein in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

What is claimed is:

1. A method for inhibiting interaction of a Plasmodium cysteine-rich protective antigen (CyRPA) with a cell that is capable of being infected by a Plasmodium pathogen, the method comprising, consisting or consisting essentially of contacting the CyRPA with a ligand of the CyRPA which when bound to the CyRPA inhibits interaction of the CyRPA with the cell.

2. A method for treating or inhibiting the development of a Plasmodium pathogen infection in a subject, the method comprising, consisting or consisting essentially of administering to the subject an effective amount of a ligand of a cysteine-rich protective antigen (CyRPA) expressed by the Plasmodium pathogen which ligand, when bound to the CyRPA, inhibits interaction of the CyRPA with a cell that is capable of being infected by the Plasmodium pathogen, to thereby treat or inhibit the development of the Plasmodium pathogen infection in the subject.

3. The method of claim 1 or claim 2, wherein the CyRPA interacts with a glycan expressed by the cell when the CyRPA is not bound to the ligand and wherein when the CyRPA is bound to the ligand, the ligand inhibits interaction of the CyRPA with the glycan.

4. The method of claim 3, wherein the glycan comprises an α2-6-linked sialic acid.

5. The method of claim 4, wherein the glycan comprises α2-6-Neu5Ac.

6. The method of claim 4 or claim 5, wherein the glycan comprises α2-6-sialyllactosamine (Neu5Ac-α-(2-6)-Gal-β-(1-4)-GlcNAc).

7. The method of any one of claims 1 to 6, wherein the cell is an erythrocyte or hepatocyte.

8. The method of any one of claims 1 to 7, wherein the Plasmodium pathogen is a drug-resistant Plasmodium pathogen.

9. The method of any one of claims 1 to 8, wherein the Plasmodium pathogen is a multidrug-resistant Plasmodium pathogen.

10. The method of any one of claims 2 to 9, wherein the CyRPA ligand is administered concurrently with an ancillary agent (e.g., an antimalarial agent or adjunctive therapy for improving efficacy, or reducing one or more malarial disease-associated complications).

11. A composition for use in therapy or prophylaxis of a Plasmodium pathogen infection in a subject, the composition comprising, consisting or consisting essentially of a ligand of a P. falciparum CyRPA which, when bound to the CyRPA, inhibits interaction of the CyRPA with an α2-6-linked sialic acid of an α2-6-linked sialic acid-expressing cell, and optionally a pharmaceutically acceptable carrier.

12. A kit for use in treating a Plasmodium pathogen infection in a subject, the kit comprising: a ligand of a Plasmodium pathogen CyRPA which, when bound to the CyRPA, inhibits interaction of the CyRPA with an α2-6-linked sialic acid of an α2-6-linked sialic acid-expressing cell, and optionally instructional material for performing the treatment.

13. The kit for use according to claim 12, further comprising an ancillary agent useful for the treatment of the Plasmodium pathogen infection (e.g., an antimalarial agent or adjunctive therapy for improving efficacy, or reducing one or more malarial disease-associated complications).

14. The method of any one of claims 1 to 10, or the composition of claim 11, or the kit for use of claim 12 or claim 13, wherein the CyRPA ligand is selected from the compounds listed in TABLE 1.
TABLE 1

| | |
|---|---|
| 1. A compound of formula (I): | |
| 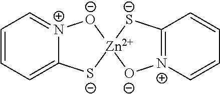 | (I) |
| (Zinc Pyrithione) | |
| 2. A compound of formula (II): | |
| 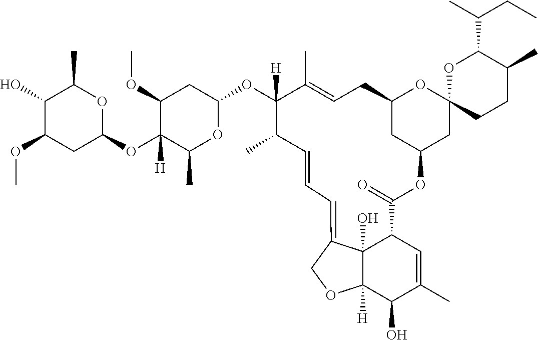 [SMILES: CCC(C)[C@H]1O[C@]2(CC[C@@H]1C)C[C@@H]1C[C@@H](C/C=C(\C)[C@@H](O[C@H]3C[C@H](OC)[C@@H](O[C@H]4C[C@@H](OC)[C@H](O)[C@@H](C)O4)[C@H](C)O3)[C@@H](C)/C=C/C=C3\CO[C@@H]4[C@H](O)C(C)=C[C@@H](C(=O)O1)[C@]34O)O2] | (II) |
| (Ivermectin) | |
| 3. A compound of formula (III): | |
| 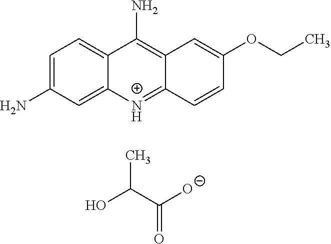 | (III) |
| (RIVANOL) | |

15. A method for treating or inhibiting the development of a Plasmodium pathogen infection in a subject the method comprising, consisting or consisting essentially of administering to the subject an effective amount of a compound of formula (I): 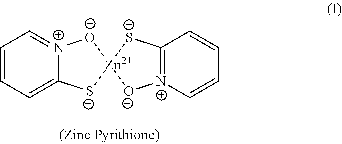
to thereby treat or inhibit the development of the Plasmodium pathogen infection in the subject.

16. A method for treating a Plasmodium pathogen infection in a subject the method comprising, consisting or consisting essentially of administering to the subject an effective amount of a compound of formula (II):

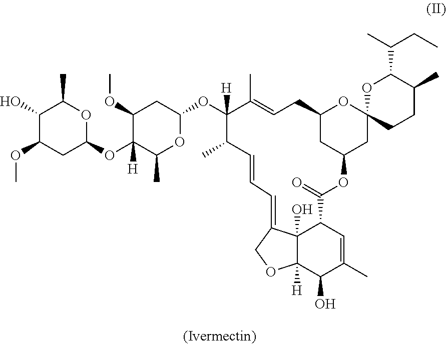 [SMILES: CCC(C)[C@H]1O[C@]2(CC[C@@H]1C)C[C@@H]1C[C@@H](C/C=C(\C)[C@@H](O[C@H]3C[C@H](OC)[C@@H](O[C@H]4C[C@@H](OC)[C@H](O)[C@@H](C)O4)[C@H](C)O3)[C@@H](C)/C=C/C=C3\CO[C@@H]4[C@H](O)C(C)=C[C@@H](C(=O)O1)[C@]34O)O2]

17. A method for treating or inhibiting the development of a Plasmodium pathogen infection in a subject the method comprising, consisting or consisting essentially of administering to the subject an effective amount of a compound of formula (III):

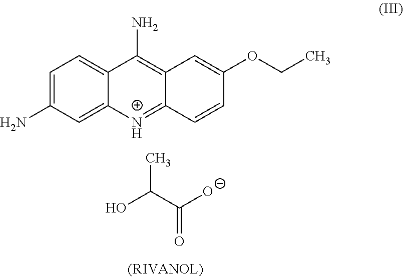

18. The method of any one of claims 15 to 17, wherein the Plasmodium pathogen is a drug-resistant Plasmodium pathogen, suitably a multidrug-resistant Plasmodium pathogen.

19. The method of any one of claims 15 to 18, comprising identifying the subject as being infected with a drug-resistant Plasmodium pathogen.

20. The method of any one of claims 15 to 19, comprising administering the compound after the subject has been administered at least one antimalarial drug to which the Plasmodium pathogen is resistant.
